(19) United States
(12) Patent Application Publication
(10) Pub. No.: US 2011/0104786 A1
(43) Pub. Date: May 5, 2011

(54) USE AND PRODUCTION OF NEUTRAL METALLOPROTEASES IN A SERINE PROTEASE-FREE BACKGROUND

(76) Inventors: Anita Van Kimmenade, San Bruno, CA (US); Edwin Lee, San Francisco, CA (US); Andrew Shaw, San Francisco, CA (US); Louise Wallace, Palo Alto, CA (US)

(21) Appl. No.: 12/681,613

(22) PCT Filed: Oct. 6, 2008
(86) PCT No.: PCT/US08/78942

§ 371 Date: Dec. 23, 2010

Related U.S. Application Data
(60) Provisional application No. 60/984,040, filed on Oct. 31, 2007.

(51) Int. Cl. C12N 9/54; C12S 9/00; D06M 16/00; C12N 1/21; C12N 1/20; C11D 3/386

(52) U.S. Cl. 435/221; 435/264; 435/263; 435/252.31; 435/252.5; 510/392

(57) ABSTRACT

The present invention provides methods and compositions comprising at least one neutral metalloprotease enzyme in the relative absence of serine protease enzyme contaminants. In some embodiments, the neutral metalloprotease finds use in cleaning and other applications. In some particularly preferred embodiments, the present invention provides methods and composi-tions comprising Bacillus strains engineered to be deficient in multiple serine proteases, and their use in production of recombinant neutral metalloprotease(s).

### RELATED APPLICATIONS

The present application claims priority to U.S. Provisional Patent Application Serial No. 60/984,040, entitled “Use and Production Of Neutral Metalloproteases In A Serine Protease-Free Background”, filed Oct. 31, 2007.

### FIELD OF THE INVENTION

The present invention provides methods and compositions comprising at least one neutral metalloprotease enzyme in the relative absence of serine protease enzyme contaminants. In some embodiments, the neutral metalloprotease finds use in cleaning and other applications. In some particularly preferred embodiments, the present invention provides methods and compositions comprising Bacillus strains engineered to be deficient in multiple serine proteases, and their use in production of recombinant neutral metalloprotease(s).

### BACKGROUND OF THE INVENTION

Members of the genus Bacillus are Gram-positive bacteria that secrete a number of industrially useful enzymes, which can be produced cheaply in high volume by fermentation. B. subtilis and other species of Bacillus produce multiple proteases that are classified according to their function and position of cleavage. Two examples include the acid proteases that cleave peptide bonds at acidic pHs, and the serine proteases that cleave the peptide bond of serine.

Given the large number of proteases present within bacterial cells and their functional diversity, it is highly unlikely that wild type or naturally occurring mutant strains will be isolated, which produce a single type of protease. Likewise, known protease purification methods are hampered by their reliance on biochemical properties that are common to multiple proteases. This is particularly problematic when the protease of interest is susceptible to degradation by protease contaminants of the Bacillus production strain.

Thus, there remains a need in the art for compositions and methods suitable for production of a heterologous protease of interest in a host strain lacking detrimental endogenous protease activity. In particular, compositions and methods for production of recombinant neutral metalloproteases in a serine protease-free background are desirable.

### SUMMARY OF THE INVENTION

The present invention provides methods and compositions comprising at least one neutral metalloprotease enzyme in the relative absence of serine protease enzyme contaminants. In some embodiments, the neutral metalloprotease finds use in cleaning and other applications. In some particularly preferred embodiments, the present invention provides methods and compositions comprising Bacillus strains engineered to be deficient in multiple serine proteases, and their use in production of recombinant neutral metalloprotease(s).

The present invention provides methods comprising: providing a Bacillus host cell lacking an endogenous serine alkaline protease enzyme (AprE), an endogenous extracellular neutral metalloprotease enzyme (NprE), and an endogenous minor extracellular serine protease enzyme (Vpr); transforming the Bacillus host cell with a nucleic acid encoding a heterologous NprE enzyme in operable combination with a promoter; and cultivating the transformed host cell under conditions suitable for the production of the heterologous NprE enzyme. In some embodiments, the methods further comprise the step of harvesting the produced heterologous NprE enzyme. In preferred embodiments, the Bacillus is B. subtilis, and in particularly preferred embodiments the B. subtilis is a BG6100 strain (ΔaprE, ΔnprE, Δvpr, oppA, ΔspoIIE, degUHy32, ΔamyE::(xylR,pxylA-comK)). In additional embodiments, the Bacillus host cell further lacks an endogenous minor extracellular serine protease enzyme (Epr). In preferred embodiments, the Bacillus is B. subtilis, and in particularly preferred embodiments the B. subtilis is a BG6101 strain (ΔaprE, ΔnprE, Δepr, Δvpr, oppA, ΔspoIIE, degUHy32, ΔamyE::(xylR,pxylA-comK)). In additional embodiments, the Bacillus host cell further lacks one or both of an endogenous major intracellular serine protease enzyme (IspA), and an endogenous bacillopeptidase F enzyme (Bpr). In preferred embodiments, the Bacillus is B. subtilis, and in particularly preferred embodiments the B. subtilis is a BG6000 strain (ΔaprE, ΔnprE, Δepr, ΔispA, Δbpf, Δvpr, oppA, ΔspoIIE, degUHy32, ΔamyE::(xylR,pxylA-comK)). In additional embodiments, the Bacillus host cell further lacks one or both of an endogenous cell wall associated protease enzyme (WprA), and an endogenous extracellular metalloprotease enzyme (Mpr). In preferred embodiments, the Bacillus is B. subtilis, and in particularly preferred embodiments the B. subtilis is a BG6003 strain (ΔaprE, ΔnprE, Δepr, ΔispA, Δbpf, Δvpr, ΔwprA, Δmpr-ybfJ, oppA, ΔspoIIE, degUHy32, ΔamyE::(xylR,pxylA-comK)). The present invention provides methods in which the heterologous NprE enzyme is a Bacillus amyloliquefaciens NprE enzyme or a variant thereof. In some embodiments, the Bacillus amyloliquefaciens NprE variant has an amino acid sequence comprising a substitution in at least one position (one, two three, four or five) selected from the group equivalent to positions 1, 3, 4, 5, 6, 11, 12, 13, 14, 16, 21, 23, 24, 25, 31, 32, 33, 35, 36, 38, 44, 45, 46, 47, 48, 49, 50, 51, 54, 55, 58, 59, 60, 61, 62, 63, 65, 66, 69, 70, 76, 85, 86, 87, 88, 90, 91, 92, 96, 97, 98, 99, 100, 102, 109, 110, 111, 112, 113, 115, 117, 119, 127, 128, 129, 130, 132, 135, 136, 137, 138, 139, 140, 146, 148, 151, 152, 153, 154, 155, 157, 158, 159, 161, 162, 169, 173, 178, 179, 180, 181, 183, 184, 186, 190, 191, 192, 196, 198, 199, 200, 202, 203, 204, 205, 210, 211, 214, 215, 216, 217, 218, 219, 220, 221, 222, 223, 224, 228, 229, 237, 239, 240, 243, 244, 245, 248, 252, 253, 260, 261, 263, 264, 265, 267, 269, 270, 273, 277, 280, 282, 283, 284, 285, 286, 288, 289, 290, 292, 293, 296, 297, and 299 of the amino acid sequence set forth as SEQ ID NO:3. In some preferred embodiments, the Bacillus amyloliquefaciens NprE variant has an amino acid sequence comprising at least one substitution (one, two three, four or five substitutions) selected from T4C, T4E, T4H, T4I, T4K, T4L, T4M, T4N, T4P, T4R, T4S, T4V, T4W, T4Y, G12D, G12E, G12I, G12K, G12L, G12M, G12Q, G12R, G12T, G12V, G12W, K13A, K13C, K13D, K13E, K13F, K13G, K13H, K13I, K13L, K13M, K13N, K13Q, K13S, K13T, K13V, K13Y, T14F, T14G, T14H, T14I, T14K, T14L, T14M, T14P, T14Q, T14R, T14S, T14V, T14W, T14Y, S23A, S23D, S23F, S23G, S23I, S23K, S23L, S23M, S23N, S23P, S23Q, S23R, S23S, S23T, S23V, S23W, S23Y, G24A, G24D, G24F, G24G, G24H, G24I, G24K, G24L, G24M, G24N, G24P, G24R, G24S, G24T, G24V, G24W, G24Y, K33H, Q45C, Q45D, Q45E, Q45F, Q45H, Q45I, Q45K, Q45L, Q45M, Q45N, Q45P, Q45R, Q45T, Q45W, N46A, N46C, N46E, N46F, N46G, N46H, N46I, N46K, N46L, N46M, N46P, N46Q, N46R, N46S, N46T, N46V, N46W, N46Y, R47E, R47K, R47L, R47M, R47Q, R47S, R47T, Y49A, Y49C, Y49D, Y49E, Y49F, Y49H, Y49I, Y49K, Y49L, Y49N, Y49R, Y49S, Y49T, Y49V, Y49W, N50D, N50F, N50G, N50H, N50I, N50K, N50L, N50M, N50P, N50Q, N50R, N50W, N50Y, T54C, T54D, T54E, T54F, T54G, T54H, T54I T54K, T54L, T54M, T54N, T54P, T54Q, T54R, T54S, T54V, T54W, T54Y, S58D, S58H, 5581, S58L, S58N, 558P, S58Q, T59A, T59C, T59E, T59G, T59H, T59I, T59K, T59L T59M, T59N, T59P, T59Q, T59R, T59S, T59V, T59W, T60D, T60F, T60I, T60K, T60L, T60N, T60Q, T60R, T60V, T60W, T60Y, T65C, T65E, T65F, T65H, T65I, T65K, T65L, T65M, T65P, T65Q, T65R, T65V, T65Y, S66C, S66D, S66E, S66F, S66H, 5661, S66K, S66L, S66N, S66P, S66Q, S66R, S66T, S66V, S66W, S66Y, Q87A, Q87D, Q87E, Q87H, Q87I, Q87K, Q87L, Q87M, Q87N, Q87R, Q87S, Q87T, Q87V, Q87W, N90C, N90D, N90E, N90F, N90G, N90H, N90K, N90L, N90R, N90T, N96G, N96H, N96K, N96R, K97H, K97Q, K97W, K100A, K100D, K100E, K100F, K100H, K100N, K100P, K100Q, K100R, K1005, K100V, K100Y, R110A, R110C, R110E, R110H, R110K, R110L, R110M, R110N, R110Q, R110S, R110Y, D119E, D119H, D1191, D119L, D119Q, D119R, D1195, D119T, D119V, D119W, G128C, G128F, G128H, G128K, G128L, G128M, G128N, G128Q, G128R, G128W, G128Y, S129A, S129C, S129D, S129F, S129G, S129H, S129I, S129K, S129L, S129M, S129Q, S129R, S129T, S129V, S129W, S129Y, F130I, F130K, F130L, F130M, F130Q, F130R, F130T, F130V, F130Y, 5135P, G136I, G136L, G136P, G136V, G136W, G136Y, S137A, M138I, M138K, M138L, M138Q, M138V, D139A, D139C, D139E, D139G, D139H, D139I, D139K, D139L, D139M, D139P, D139R, D139S, D139V, D139W, D139Y, V140C, Q151I, E152A, E152C, E152D, E152F, E152G, E152H, E152L, E152M, E152N, E152R, E152S, E152W, N155D, N155K, N155Q, N155R, D178A, D178C, D178G, D178H, D178K, D178L, D178M, D178N, D178P, D178Q, D178R, D178S, D178T, D178V, D178W, D178Y, T179A, T179F, T179H, T179I, T179K, T179L, T179M, T179N, T179P, T179Q, T179R, T179S, T179V, T179W, T179Y, E186A, E186C, E186D, E186G, E186H, E186K, E186L, E186M, E186N, E186P, E186Q, E186R, E186S, E186T, E186V, E186W, E186Y, V190H, V190I, V190K, V190L, V190Q, V190R, S191F, S191G, S191H, S1911, S191K, S191L, S191N, S191Q, S191R, S191W, L198M, L198V, S199C, S199D, S199E, S199F, S199I, S199K, S199L, S199N, S199Q, S199R, S199V, Y204H, Y204T, G205F, G205H, G205L, G205M, G205N, G205R, G205S, G205Y, K211A, K211C, K211D, K211G, K211M, K211N, K211Q, K211R, K211S, K211T, K211V, K214A, K214C, K214E, K214I, K214L, K214M, K214N, K214Q, K214R, K214S, K214V, L216A, L216C, L216F, L216H, L216Q, L216R, L216S, L216Y, N218K, N218P, T219D, D220A, D220E, D220H, D220K, D220N, D220P, A221D, A221E, A221F, A221I, A221K, A221L, A221M, A221N, A221S, A221V, A221Y, G222C, G222H, G222N, G222R, Y224F, Y224H, Y224N, Y224R, T243C, T243G, T243H, T243I, T243K, T243L, T243Q, T243R, T243W, T243Y, K244A, K244C, K244D, K244E, K244F, K244G, K244L, K244M, K244N, K244Q, K244S, K244T, K244V, K244W, K244Y, V260A, V260D, V260E, V260G, V260H, V260I, V260K, V260L, V260M, V260P, V260Q, V260R V260S, V260T, V260W, V260Y, Y261C, Y261F, Y261I, Y261L, T263E, T263F, T263H, T263I, T263L, T263M, T263Q, T263V, T263W, T263Y, S265A, S265C, S265D, S265E, S265K, S265N, S265P, S265Q, S265R, S265T, S265V, S265W, K269E, K269F, K269G, K269H, K269I, K269L, K269M, K269N, K269P, K269Q, K269S, K269T, K269V, K269W, K269Y, A273C, A273D, A273H, A273I, A273K, A273L, A273N, A273Q, A273R, A273Y, R280A, R280C, R280D, R280E, R280F, R280G, R280H, R280K, R280L, R280M, R280S, R280T, R280V, R280W, R280Y, L282F, L282G, L282H, L282I, L282K, L282M, L282N, L282Q, L282R, L282V, L282Y, S285A, S285C, S285D, S285E, S285K, S285P, S285Q, S285R, S285W, Q286A, Q286D, Q286E, Q286K, Q286P, Q286R, A289C, A289D, A289E, A289K, A289L, A289R, A293C, A293R, N296C, N296D, N296E, N296K, N296R, N296V, A297C, A297K, A297N, A297Q, A297R, and G299N. In some particularly preferred embodiments, the substitutions comprise multiple mutations selected from S129I/F130L/D220P, M138L/V190I/D220P, and S120I/F130L/M138L/V190I/D220P. In some preferred embodiments, the neutral metalloprotease has at least about 45% amino acid identity with the neutral metalloprotease comprising the amino acid sequence set forth as SEQ ID NO:3. Also provided by the present invention are compositions comprising the heterologous NprE enzyme produced by the methods of the present invention.

Moreover the present invention provides compositions comprising an isolated Bacillus neutral metalloprotease enzyme (NprE) or variant thereof, wherein the composition is essentially devoid of Bacillus serine protease enzyme (AprE) contamination. In some preferred embodiments, the AprE contamination comprises less than about 1% by weight as compared to the NprE or variant thereof. In some preferred embodiments, the AprE contamination comprises less than 0.50 U/ml serine protease activity, preferably less than 0.05 U/ml serine protease activity, and more preferably less than 0.005 U/ml serine protease activity. In some preferred embodiments, the composition is a cleaning composition. In some preferred embodiments, the cleaning composition is a detergent. In some particularly preferred embodiments, the composition further comprises at least one additional enzyme or enzyme derivative selected from amylases, lipases, mannanases, pectinases, cutinases, oxidoreductases, hemicellulases, and cellulases. In some embodiments, the composition comprises at least about 0.0001 weight percent of the neutral metalloprotease variant, and preferably from about 0.001 to about 0.5 weight percent of the neutral metalloprotease variant. In some embodiments, the composition further comprises at least one adjunct ingredient. The present invention provides compositions further comprising a sufficient amount of a pH modifier to provide the composition with a neat pH of from about 3 to about 5, the composition being essentially free of materials that hydrolyze at a pH of from about pH 3 to about pH 5. In some embodiments, the materials that hydrolyze at a pH of from about pH 3 to about pH 5 comprise at least one surfactant. In a subset of these embodiments, the surfactant is a sodium alkyl sulfate surfactant comprising an ethylene oxide moiety. In some embodiments, the composition is a liquid. The present invention further provides methods of cleaning, comprising the step of contacting a surface and/or an article comprising a fabric with a cleaning composition of the present invention. In some embodiments, the methods further comprise the step of rinsing the surface and/or material after contacting the surface or material with the cleaning composition. In other embodiments, the composition is an animal feed composition comprising an isolated neutral metalloprotease variant. In alternative embodiments, the composition is a textile processing composition comprising an isolated neutral metalloprotease variant. In additional embodiments, the composition is a leather processing composition comprising an isolated neutral metalloprotease variant.

Also provided by the present invention are isolated Bacillus host cells lacking an endogenous serine alkaline protease enzyme (AprE), an endogenous extracellular neutral metalloprotease enzyme (NprE), and an endogenous minor extracellular serine protease enzyme (Vpr), wherein the host cell is transformed with a nucleic acid encoding a heterologous NprE enzyme in operable combination with a promoter. In some embodiments, the Bacillus is B. subtilis, while in some preferred embodiments the B. subtilis is a BG6100 strain (ΔaprE, ΔnprE, Δvpr, oppA, ΔspoIIE, degUHy32, ΔamyE::(xylR,pxylA-comK)). In some embodiments, the Bacillus host cell further lacks an endogenous minor extracellular serine protease enzyme (Epr). In some embodiments, the Bacillus is B. subtilis, while in some preferred embodiments the B. subtilis is a BG6101 strain (ΔaprE, ΔnprE, Δepr, Δvpr, oppA, ΔspoIIE, degUHy32, ΔamyE::(xylR,pxylA-comK)). In additional embodiments, the Bacillus host cell further lacks one or both of an endogenous major intracellular serine protease enzyme (IspA), and an endogenous bacillopeptidase F enzyme (Bpr). In some embodiments, the Bacillus is B. subtilis, while in some preferred embodiments the B. subtilis is a BG6000 strain (ΔaprE, ΔnprE, Δepr, ΔispA, Δbpf, Δvpr, oppA, ΔspoIIE, degUHy32, ΔamyE::(xylR,pxylA-comK)). In additional embodiments, the Bacillus host cell further lacks one or both of an endogenous cell wall associated protease enzyme (WprA), and an endogenous extracellular metalloprotease enzyme (Mpr). In some embodiments, the Bacillus is B. subtilis, while in some preferred embodiments the B. subtilis is a BG6003 strain (ΔaprE, ΔnprE, Δepr, ΔispA, Δbpf, Δvpr, ΔwprA, Δmpr-ybfJ, oppA, ΔspoIIE, degUHy32, ΔamyE::(xylR,pxylA-comK)). In still further preferred embodiments, the heterologous NprE enzyme is a Bacillus amyloliquefaciens NprE enzyme or a variant thereof.

The present invention provides an isolated Bacillus host cell lacking an endogenous serine alkaline protease enzyme (AprE), an endogenous extracellular neutral metalloprotease enzyme (NprE), an endogenous minor extracellular serine protease enzyme (Vpr), an endogenous minor extracellular serine protease enzyme (Epr), an endogenous major intracellular serine protease enzyme (IspA), and an endogenous bacillopeptidase F enzyme (Bpr). In some embodiments, the Bacillus is B. subtilis, while in some preferred embodiments the B. subtilis is a BG6000 strain (ΔaprE, ΔnprE, Δepr, ΔispA, Δbpf, Δvpr, oppA, ΔspoIIE, degUHy32, ΔamyE::(xylR,pxylA-comK)). In other embodiments, the present invention provides an isolated Bacillus host cell lacking an endogenous serine alkaline protease enzyme (AprE), an endogenous extracellular neutral metalloprotease enzyme (NprE), an endogenous minor extracellular serine protease enzyme (Vpr), an endogenous minor extracellular serine protease enzyme (Epr), an endogenous major intracellular serine protease enzyme (IspA), an endogenous bacillopeptidase F enzyme (Bpr), an endogenous cell wall associated protease enzyme (WprA), and an endogenous extracellular metalloprotease enzyme (Mpr). In some embodiments, the Bacillus is B. subtilis, while in some preferred embodiments the B. subtilis is a BG6003 strain (ΔaprE, ΔnprE, Δepr, ΔispA, Δbpf, Δvpr, ΔwprA, Δmpr-ybfJ, oppA, ΔspoIIE, degUHy32, ΔamyE::(xylR,pxylA-comK)).

### BRIEF DESCRIPTION OF THE DRAWINGS

FIG. 1 illustrates a general scheme for creation of Bacillus host strains bearing deletions in endogenous protease genes. This figure shows an exemplary strategy for deletion of the Bacillus subtilis wall protease (wprA) gene, through the use of the antibiotics spectinomycin and kanamycin, and plasmids bearing spectinomycin-resistance (spec) and kanamycin-resistance (kan) genes.

FIG. 2 provides maps of plasmids used as PCR templates. Panel A provides a map of plasmid pJHT. Panel B provides a map of plasmid pUBnprE.

FIG. 3 provides a schematic of an exemplary spliced-overlap-extension (SOE) reaction used to prepare a nucleic acid comprising a B. amyloliquefaciens nprE coding sequence in operable combination with an aprE promoter sequence.

FIG. 4 provides a DNA sequence (SEQ ID NO:12) of the nucleic acid produced by the SOE reaction of the previous figure. Lower-case indicates the aprE promoter, lower-case with a single underline indicates the B. amyloliquefaciens nprE signal sequence, lower-case with double underlines indicates the B. amyloliquefaciens nprE pro sequence, and upper-case indicates the mature B. amyloliquefaciens nprE sequence.

FIG. 5 provides the results obtained by assessment of serine protease contamination in fermentation broths of Bacillus protease knock-out strains. Serine protease expression and activity was measured by SDS-PAGE analysis and AAPF assay, respectively. The 20-30 kDa and 100 kDa protein bands were identified by N-terminal sequencing, revealing that the 100 kDa protease corresponds to the minor extracellular protease Vpr (Sloma et al., J Bacteriol, 173:21, 6889, 1991).

FIG. 6 provides densitometry graphs of lanes of the gel of the previous figure. The graph corresponding to the fermentation broth of the two (2) protease deletion strain is shown on the left, while the graph corresponding to the fermentation broth of the eight (8) protease deletion strain is shown on the right.

FIG. 7 illustrates the construction of a protease gene deletion plasmid by PCR amplification of homologous upstream and downstream chromosomal DNA with convenient restriction sites introduced at the primer termini (See e.g., FIG. 7).

FIG. 8 provides a map of the pLoxSpec plasmid.

FIG. 9 provides a schematic of the linearized plasmid bearing the upstream chromosomal DNA-Spec-loxP-downstream chromosomal DNA cassette.

FIG. 10 provides a map of the pCRM-Ts Phleo plasmid.

### GENERAL DESCRIPTION OF THE INVENTION

The present invention provides methods and compositions comprising at least one neutral metalloprotease enzyme in the relative absence of serine protease enzyme contaminants. In some embodiments, the neutral metalloprotease finds use in cleaning and other applications. In some particularly preferred embodiments, the present invention provides methods and compositions comprising Bacillus strains engineered to be deficient in multiple serine proteases, and their use in production of recombinant neutral metalloprotease(s).

Unless otherwise indicated, the practice of the present invention involves conventional techniques commonly used in molecular biology, microbiology, and recombinant DNA, which are within the skill of the art. Such techniques are known to those of skill in the art and are described in numerous texts and reference works (See e.g., Sambrook et al., “Molecular Cloning: A Laboratory Manual,” Second Edition, Cold Spring Harbor, 1989; and Ausubel et al., “Current Protocols in Molecular Biology,” 1987). All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

Also, as used herein, the singular “a”, “an” and “the” includes the plural reference unless the context clearly indicates otherwise. Numeric ranges are inclusive of the numbers defining the range. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Furthermore, the headings provided herein are not limitations of the various aspects or embodiments of the invention, which can be had by reference to the specification as a whole. Accordingly, the terms defined immediately below are more fully defined by reference to the specification as a whole. Nonetheless, in order to facilitate understanding of the invention, a number of terms are defined below.

### DEFINITIONS

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

As used herein, the terms “protease,” and “proteolytic activity” refer to a protein or peptide exhibiting the ability to hydrolyze peptides or substrates having peptide linkages Many well known procedures exist for measuring proteolytic activity (Kalisz, “Microbial Proteinases,” In: Fiechter (ed.), Advances in Biochemical Engineering/Biotechnology, 1988). For example, proteolytic activity may be ascertained by comparative assays, which analyze the respective protease's ability to hydrolyze a commercial substrate. Exemplary substrates useful in such analysis of protease or proteolytic activity, include, but are not limited to di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO 99/34011; and U.S. Pat. No. 6,376,450, both of which are incorporated herein by reference. The pNA assay (See e.g., Del Mar et al., Anal Biochem, 99:316-320, 1979) also finds use in determining the active enzyme concentration for fractions collected during gradient elution. This assay measures the rate at which p-nitroaniline is released as the enzyme hydrolyzes the soluble synthetic substrate, succinyl-alanine-alanine-proline-phenylalanine-p-nitroanilide (sAAPF-pNA). The rate of production of yellow color from the hydrolysis reaction is measured at 410 nm on a spectrophotometer and is proportional to the active enzyme concentration. In addition, absorbance measurements at 280 nm can be used to determine the total protein concentration. The active enzyme/total-protein ratio gives the enzyme purity.

As used herein, the terms “NprE protease,” and “NprE,” refer to the neutral metalloproteases described herein. In some preferred embodiments, the NprE protease is the protease designated herein as purified MULTIFECT® Neutral or PMN obtained from Bacillus amyloliquefaciens. Thus, in some embodiments, the term “PMN protease” refers to a naturally occurring mature protease derived from Bacillus amyloliquefaciens having substantially identical amino acid sequences as provided in SEQ ID NO:3. In alternative embodiments, the present invention provides portions of the NprE protease.

The term “Bacillus protease homologues” refers to naturally occurring proteases having substantially identical amino acid sequences to the mature protease derived from Bacillus amyloliquefaciens or polynucleotide sequences which encode for such naturally occurring proteases, and which proteases retain the functional characteristics of a neutral metalloprotease encoded by such nucleic acids.

As used herein, the terms “NprE variant,” and “NprE protease variant,” are used in reference to proteases that are similar to the wild-type NprE, particularly in their function, but have mutations in their amino acid sequence that make them different in sequence from the wild-type protease.

As used herein, “Bacillus ssp.” refers to all of the species within the genus “Bacillus,” which are Gram-positive bacteria classified as members of the Family Bacillaceae, Order Bacillales, Class Bacilli. The genus “Bacillus” includes all species within the genus “Bacillus,” as known to those of skill in the art, including but not limited to B. subtilis, B. licheniformis, B. lentus, B. brevis, B. stearothennophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

Related (and derivative) proteins comprise “variant proteins.” In some preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In some preferred embodiments, the number of different amino acids between variants is between 1 and 10. In some particularly preferred embodiments, related proteins and particularly variant proteins comprise at least about 35%, about 40%, about 45%, about 50%, about 55%, about 60%, about 65%, about 70%, about 75%, about 80%, about 85%, about 90%, about 95%, about 97%, about 98%, or about 99% amino acid sequence identity. Additionally, a related protein or a variant protein as used herein, refers to a protein that differs from another related protein or a parent protein in the number of prominent regions. For example, in some embodiments, variant proteins have 1, 2, 3, 4, 5, or 10 corresponding prominent regions that differ from the parent protein.

Several methods are known in the art that are suitable for generating variants of the enzymes of the present invention, including but not limited to site-saturation mutagenesis, scanning mutagenesis, insertional mutagenesis, random mutagenesis, site-directed mutagenesis, and directed-evolution, as well as various other recombinatorial approaches.

Characterization of wild-type and mutant proteins is accomplished via any means or “test” suitable and is preferably based on the assessment of properties of interest. For example, pH and/or temperature, as well as detergent and/or oxidative stability is/are determined in some embodiments of the present invention. Indeed, it is contemplated that enzymes having various degrees of stability in one or more of these characteristics (pH, temperature, proteolytic stability, detergent stability, and/or oxidative stability) will find use.

The terms “polynucleotide” and “nucleic acid”, used interchangeably herein, refer to a polymeric form of nucleotides of any length, either ribonucleotides or deoxyribonucleotides. These terms include, but are not limited to, a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The following are non-limiting examples of polynucleotides: genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In some embodiments, polynucleotides comprise modified nucleotides, such as methylated nucleotides and nucleotide analogs, uracil, other sugars and linking groups such as fluororibose and thioate, and nucleotide branches. In alternative embodiments, the sequence of nucleotides is interrupted by non-nucleotide components.

As used herein, the terms “DNA construct” and “transforming DNA” are used interchangeably to refer to DNA used to introduce sequences into a host cell or organism. The DNA may be generated in vitro by PCR or any other suitable technique(s) known to those in the art. In particularly preferred embodiments, the DNA construct comprises a sequence of interest (e.g., as an incoming sequence). In some embodiments, the sequence is operably linked to additional elements such as control elements (e.g., promoters, etc.). The DNA construct may further comprise a selectable marker. It may further comprise an incoming sequence flanked by homology boxes. In a further embodiment, the transforming DNA comprises other non-homologous sequences, added to the ends (e.g., stuffer sequences or flanks). In some embodiments, the ends of the incoming sequence are closed such that the transforming DNA forms a closed circle. The transforming sequences may be wild-type, mutant or modified. In some embodiments, the DNA construct comprises sequences homologous to the host cell chromosome. In other embodiments, the DNA construct comprises non-homologous sequences. Once the DNA construct is assembled in vitro it may be used to: 1) insert heterologous sequences into a desired target sequence of a host cell, and/or 2) mutagenize a region of the host cell chromosome (i.e., replace an endogenous sequence with a heterologous sequence), 3) delete target genes; and/or introduce a replicating plasmid into the host.

As used herein, the terms “expression cassette” and “expression vector” refer to nucleic acid constructs generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In preferred embodiments, expression vectors have the ability to incorporate and express heterologous DNA fragments in a host cell. Many prokaryotic and eukaryotic expression vectors are commercially available. Selection of appropriate expression vectors is within the knowledge of those of skill in the art. The term “expression cassette” is used interchangeably herein with “DNA construct,” and their grammatical equivalents. Selection of appropriate expression vectors is within the knowledge of those of skill in the art.

As used herein, the term “vector” refers to a polynucleotide construct designed to introduce nucleic acids into one or more cell types. Vectors include cloning vectors, expression vectors, shuttle vectors, plasmids, cassettes and the like. In some embodiments, the polynucleotide construct comprises a DNA sequence encoding the protease (e.g., precursor or mature protease) that is operably linked to a suitable prosequence (e.g., secretory, etc.) capable of effecting the expression of the DNA in a suitable host.

As used herein, the term “plasmid” refers to a circular double-stranded (ds) DNA construct used as a cloning vector, and which forms an extrachromosomal self-replicating genetic element in some eukaryotes or prokaryotes, or integrates into the host chromosome.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., “Genetics,” in Hardwood et al, (eds.), Bacillus, Plenum Publishing Corp., pages 57-72, 1989).

As used herein, the terms “transformed” and “stably transformed” refers to a cell that has a non-native (heterologous) polynucleotide sequence integrated into its genome or as an episomal plasmid that is maintained for at least two generations.

As used herein, the term “selectable marker-encoding nucleotide sequence” refers to a nucleotide sequence, which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell, which allows for ease of selection of those hosts containing the vector. Examples of such selectable markers include but are not limited to antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, preferably the marker is an antimicrobial resistant marker (e.g., amp^{R}; phleo^{R}; SpeC^{R}; kan^{R}; ery^{R}; tet^{R}; cmp^{R}; and neo^{R} (See e.g., Guerot-Fleury, Gene, 167:335-337, 1995; Palmeros et al., Gene 247:255-264, 2000; and Trieu-Cuot et al., Gene, 23:331-341, 1983). Other markers useful in accordance with the invention include, but are not limited to auxotrophic markers, such as tryptophan; and detection markers, such as β-galactosidase.

As used herein, the term “promoter” refers to a nucleic acid sequence that functions to direct transcription of a downstream gene. In preferred embodiments, the promoter is appropriate to the host cell in which the target gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein the term “gene” refers to a polynucleotide (e.g., a DNA segment) that encodes a polypeptide and includes regions preceding and following the coding regions as well as intervening sequences (introns) between individual coding segments (exons).

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “ortholog” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred. This homology is determined using standard techniques known in the art (See e.g., Smith and Waterman, Adv Appl Math, 2:482, 1981; Needleman and Wunsch, J Mol Biol, 48:443, 1970; Pearson and Lipman, Proc Natl Acad Sci USA, 85:2444, 1988; programs such as GAP, BESTFIT, FASTA, and TFASTA in the Wisconsin Genetics Software Package, Genetics Computer Group, Madison, Wis.; and Devereux et al., Nucl Acid Res, 12:387-395, 1984).

As used herein, an “analogous sequence” is one wherein the function of the gene is essentially the same as the gene based on the B. amyloliquefaciens NprE protease. Additionally, analogous genes include at least about 45%, about 50%, about 55%, about 60%, about 65%, about 70%, about 75%, about 80%, about 85%, about 90%, about 95%, about 97%, about 98%, about 99%, or about 100% sequence identity with the sequence of the B. amyloliquefaciens NprE protease. In additional embodiments more than one of the above properties applies to the sequence. Analogous sequences are determined by known methods of sequence alignment. A commonly used alignment method is BLAST, although as indicated above and below, there are other methods that also find use in aligning sequences.

One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J Mol Evol, 35:351-360, 1987). The method is similar to that described by Higgins and Sharp (Higgins and Sharp, CABIOS 5:151-153, 1989). Useful PILEUP parameters including a default gap weight of 3.00, a default gap length weight of 0.10, and weighted end gaps.

Another example of a useful algorithm is the BLAST algorithm, described by Altschul et al., (Altschul et al., J Mol Biol, 215:403-410, 1990; and Karlin et al., Proc Natl Acad Sci USA, 90:5873-5787, 1993). A particularly useful BLAST program is the WU-BLAST-2 program (See, Altschul et al., Meth Enzymol, 266:460-480, 1996). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A “% amino acid sequence identity” value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical to the nucleotide residues of the starting sequence (i.e., the sequence of interest). A preferred method utilizes the BLASTN module of WU-BLAST-2 set to the default parameters, with overlap span and overlap fraction set to 1 and 0.125, respectively.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination,” “recombining,” and generating a “recombined” nucleic acid are generally the assembly of two or more nucleic acid fragments wherein the assembly gives rise to a chimeric gene.

In a preferred embodiment, mutant DNA sequences are generated with site saturation mutagenesis in at least one codon. In another preferred embodiment, site saturation mutagenesis is performed for two or more codons. In a further embodiment, mutant DNA sequences have more than about 50%, more than about 55%, more than about 60%, more than about 65%, more than about 70%, more than about 75%, more than about 80%, more than about 85%, more than about 90%, more than about 95%, or more than about 98% homology with the wild-type sequence. In alternative embodiments, mutant DNA is generated in vivo using any known mutagenic procedure such as, for example, radiation, nitrosoguanidine and the like. The desired DNA sequence is then isolated and used in the methods provided herein.

As used herein, the term “target sequence” refers to a DNA sequence in the host cell that encodes the sequence where it is desired for the incoming sequence to be inserted into the host cell genome. In some embodiments, the target sequence encodes a functional wild-type gene or operon, while in other embodiments the target sequence encodes a functional mutant gene or operon, or a non-functional gene or operon.

As used herein, a “flanking sequence” refers to any sequence that is either upstream or downstream of the sequence being discussed (e.g., for genes A-B-C, gene B is flanked by the A and C gene sequences). In a preferred embodiment, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. In some embodiments, a flanking sequence is present on only a single side (either 3′ or 5′), while in preferred embodiments, it is present on each side of the sequence being flanked.

As used herein, the term “sniffer sequence” refers to any extra DNA that flanks homology boxes (typically vector sequences). However, the term encompasses any non-homologous DNA sequence. Not to be limited by any theory, a stuffer sequence provides a noncritical target for a cell to initiate DNA uptake.

As used herein, the terms “amplification” and “gene amplification” refer to a process by which specific DNA sequences are disproportionately replicated such that the amplified gene becomes present in a higher copy number than was initially present in the genome. In some embodiments, selection of cells by growth in the presence of a drug (e.g., an inhibitor of an inhibitable enzyme) results in the amplification of either the endogenous gene encoding the gene product required for growth in the presence of the drug or by amplification of exogenous (i.e., input) sequences encoding this gene product, or both.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “co-amplification” refers to the introduction into a single cell of an amplifiable marker in conjunction with other gene sequences (i.e., comprising one or more non-selectable genes such as those contained within an expression vector) and the application of appropriate selective pressure such that the cell amplifies both the amplifiable marker and the other, non-selectable gene sequences. The amplifiable marker may be physically linked to the other gene sequences or alternatively two separate pieces of DNA, one containing the amplifiable marker and the other containing the non-selectable marker, may be introduced into the same cell.

As used herein, the terms “amplifiable marker,” “amplifiable gene,” and “amplification vector” refer to a gene or a vector encoding a gene, which permits the amplification of that gene under appropriate growth conditions.

“Template specificity” is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (See e.g., Kacian et al., Proc Natl Acad Sci USA 69:3038, 1972) and other nucleic acids are not replicated by this amplification enzyme. Similarly, in the case of T7 RNA polymerase, this amplification enzyme has a stringent specificity for its own promoters (See, Chamberlin et al., Nature 228:227, 1970). In the case of T4 DNA ligase, the enzyme will not ligate the two oligonucleotides or polynucleotides, where there is a mismatch between the oligonucleotide or polynucleotide substrate and the template at the ligation junction (See, Wu and Wallace, Genomics 4:560, 1989). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “amplifiable nucleic acid” refers to nucleic acids, which may be amplified by any amplification method. It is contemplated that “amplifiable nucleic acid” will usually comprise “sample template.”

As used herein, the term “sample template” refers to nucleic acid originating from a sample, which is analyzed for the presence of “target” (defined below). In contrast, “background template” is used in reference to nucleic acid other than sample template, which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “RT-PCR” refers to the replication and amplification of RNA sequences. In this method, reverse transcription is coupled to PCR, most often using a one enzyme procedure in which a thermostable polymerase is employed, as described in U.S. Pat. No. 5,322,770, herein incorporated by reference. In RT-PCR, the RNA template is converted to cDNA due to the reverse transcriptase activity of the polymerase, and then amplified using the polymerizing activity of the polymerase (i.e., as in other PCR methods).

As used herein, the terms “restriction endonucleases” and “restriction enzymes” refer to bacterial enzymes, each of which cut double-stranded DNA at or near a specific nucleotide sequence.

A “restriction site” refers to a nucleotide sequence recognized and cleaved by a given restriction endonuclease and is frequently the site for insertion of DNA fragments. In certain embodiments of the invention restriction sites are engineered into the selective marker and into 5′ and 3′ ends of the DNA construct.

As used herein, the term “chromosomal integration” refers to the process whereby an incoming sequence is introduced into the chromosome of a host cell. The homologous regions of the transforming DNA align with homologous regions of the chromosome. Subsequently, the sequence between the homology boxes is replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the Bacillus chromosome. Subsequently, the indigenous chromosomal region is deleted by the DNA construct in a double crossover (i.e., homologous recombination).

“Homologous recombination” means the exchange of DNA fragments between two DNA molecules or paired chromosomes at the site of identical or nearly identical nucleotide sequences. In a preferred embodiment, chromosomal integration is homologous recombination. “Homologous sequences” as used herein means a nucleic acid or polypeptide sequence having about 100%, about 99%, about 98%, about 97%, about 96%, about 95%, about 94%, about 93%, about 92%, about 91%, about 90%, about 88%, about 85%, about 80%, about 75%, or about 70% sequence identity to another nucleic acid or polypeptide sequence when optimally aligned for comparison. In some embodiments, homologous sequences have between about 85% and about 100% sequence identity, while in other embodiments there is between about 90% and about 100% sequence identity, and in more preferred embodiments, there is about 95% and about 100% sequence identity.

As used herein “amino acid” refers to peptide or protein sequences or portions thereof. The terms “protein,” “peptide,” and “polypeptide” are used interchangeably.

As used herein, the term “heterologous protein” refers to a protein or polypeptide that does not naturally occur in the host cell. Examples of heterologous proteins include enzymes such as hydrolases including proteases. In some embodiments, the gene encoding the proteins are naturally occurring genes, while in other embodiments, mutated and/or synthetic genes are used.

As used herein, “homologous protein” refers to a protein or polypeptide native or naturally occurring in a cell. In preferred embodiments, the cell is a Gram-positive cell, while in particularly preferred embodiments the cell is a Bacillus host cell. In alternative embodiments, the homologous protein is a native protein produced by other organisms, including but not limited to E. coli, Streptomyces, Trichoderma, and Aspergillus. The invention encompasses host cells producing the homologous protein via recombinant DNA technology.

As used herein, an “operon region” comprises a group of contiguous genes that are transcribed as a single transcription unit from a common promoter, and are thereby subject to co-regulation. In some embodiments, the operon includes a regulator gene. In most preferred embodiments, operons that are highly expressed as measured by RNA levels, but have an unknown or unnecessary function are used.

As used herein, an “antimicrobial region” is a region containing at least one gene that encodes an antimicrobial protein.

A polynucleotide is said to “encode” an RNA or a polypeptide if, in its native state or when manipulated by methods known to those of skill in the art, it can be transcribed and/or translated to produce the RNA, the polypeptide or a fragment thereof. The anti-sense strand of such a nucleic acid is also said to encode the sequences.

As is known in the art, a DNA can be transcribed by an RNA polymerase to produce RNA, but an RNA can be reverse transcribed by reverse transcriptase to produce a DNA. Thus a DNA can encode a RNA and vice versa.

The term “regulatory segment” or “regulatory sequence” or “expression control sequence” refers to a polynucleotide sequence of DNA that is operatively linked with a polynucleotide sequence of DNA that encodes the amino acid sequence of a polypeptide chain to effect the expression of the encoded amino acid sequence. The regulatory sequence can inhibit, repress, or promote the expression of the operably linked polynucleotide sequence encoding the amino acid.

“Host strain” or “host cell” refers to a suitable host for an expression vector comprising DNA according to the present invention.

An enzyme is “overexpressed” in a host cell if the enzyme is expressed in the cell at a higher level that the level at which it is expressed in a corresponding wild-type cell.

The terms “protein” and “polypeptide” are used interchangeability herein. The 3-letter code for amino acids as defined in conformity with the IUPAC-IUB Joint Commission on Biochemical Nomenclature (JCBN) is used through out this disclosure. It is also understood that a polypeptide may be coded for by more than one nucleotide sequence due to the degeneracy of the genetic code.

A “prosequence” is an amino acid sequence between the signal sequence and mature protease that is necessary for the secretion of the protease. Cleavage of the pro sequence will result in a mature active protease.

The term “signal sequence” or “signal peptide” refers to any sequence of nucleotides and/or amino acids that participate in the secretion of the mature or precursor forms of the protein. This definition of signal sequence is a functional one, meant to include all those amino acid sequences encoded by the N-terminal portion of the protein gene, which participate in the effectuation of the secretion of protein. They are often, but not universally, bound to the N-terminal portion of a protein or to the N-terminal portion of a precursor protein. The signal sequence may be endogenous or exogenous. The signal sequence may be that normally associated with the protein (e.g., protease), or may be from a gene encoding another secreted protein. One exemplary exogenous signal sequence comprises the first seven amino acid residues of the signal sequence from Bacillus subtilis subtilisin fused to the remainder of the signal sequence of the subtilisin from Bacillus lentus (ATCC 21536).

The term “hybrid signal sequence” refers to signal sequences in which part of sequence is obtained from the expression host fused to the signal sequence of the gene to be expressed. In some embodiments, synthetic sequences are utilized.

The term “mature” form of a protein or peptide refers to the final functional form of the protein or peptide. To exemply, a mature form of the NprE protease of the present invention at least includes the amino acid sequence of SEQ ID NO:3.

The term “precursor” form of a protein or peptide refers to a mature form of the protein having a prosequence operably linked to the amino or carbonyl terminus of the protein. The precursor may also have a “signal” sequence operably linked, to the amino terminus of the prosequence. The precursor may also have additional polynucleotides that are involved in post-translational activity (e.g., polynucleotides cleaved therefrom to leave the mature form of a protein or peptide).

“Naturally occurring enzyme” refers to an enzyme having the unmodified amino acid sequence identical to that found in nature. Naturally occurring enzymes include native enzymes, those enzymes naturally expressed or found in the particular microorganism.

The terms “derived from” and “obtained from” refer to not only a protease produced or producible by a strain of the organism in question, but also a protease encoded by a DNA sequence isolated from such strain and produced in a host organism containing such DNA sequence. Additionally, the term refers to a protease that is encoded by a DNA sequence of synthetic and/or cDNA origin and which has the identifying characteristics of the protease in question. To exemplify, “proteases derived from Bacillus sp.” refers to those enzymes having proteolytic activity which are naturally-produced by Bacillus sp., as well as to neutral metalloproteases like those produced by Bacillus sp. sources but which through the use of genetic engineering techniques are produced by non-Bacillus amyloliquefaciens organisms transformed with a nucleic acid encoding said neutral metalloproteases.

A “derivative” within the scope of this definition generally retains the characteristic proteolytic activity observed in the wild-type, native or parent form to the extent that the derivative is useful for similar purposes as the wild-type, native or parent form. Functional derivatives of neutral metalloprotease encompass naturally occurring, synthetically or recombinantly produced peptides or peptide fragments having the general characteristics of the neutral metalloprotease of the present invention.

The term “functional derivative” refers to a derivative of a nucleic acid having the functional characteristics of a nucleic acid encoding a neutral metalloprotease. Functional derivatives of a nucleic acid, which encode neutral metalloprotease of the present invention encompass naturally occurring, synthetically or recombinantly produced nucleic acids or fragments and encode neutral metalloprotease characteristic of the present invention. Wild type nucleic acid encoding neutral metalloprotease according to the invention include naturally occurring alleles and homologues based on the degeneracy of the genetic code known in the art.

The term “identical” in the context of two nucleic acids or polypeptide sequences refers to the residues in the two sequences that are the same when aligned for maximum correspondence, as measured using one of the following sequence comparison or analysis algorithms.

The term “optimal alignment” refers to the alignment giving the highest percent identity score.

“Percent sequence identity,” “percent amino acid sequence identity,” “percent gene sequence identity,” and/or “percent nucleic acid/polynucloetide sequence identity,” with respect to two amino acid, polynucleotide and/or gene sequences (as appropriate), refer to the percentage of residues that are identical in the two sequences when the sequences are optimally aligned. Thus, “80% amino acid sequence identity” means that 80% of the amino acids in two optimally aligned polypeptide sequences are identical.

The phrase “substantially identical” in the context of two nucleic acids or polypeptides thus refers to a polynucleotide or polypeptide that comprising at least about 70% sequence identity, preferably at least about 75%, preferably at least about 80%, preferably at least about 85%, preferably at least about 90%, preferably at least about 95%, preferably at least about 97%, preferably at least about 98% and preferably at least about 99% sequence identity as compared to a reference sequence using the programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

The term “isolated” or “purified” refers to a material that is removed from its original environment (e.g., the natural environment if it is naturally occurring). For example, the material is said to be “purified” when it is present in a particular composition in a higher or lower concentration than exists in a naturally occurring or wild type organism or in combination with components not normally present upon expression from a naturally occurring or wild type organism. For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector, and/or such polynucleotides or polypeptides could be part of a composition, and still be isolated in that such vector or composition is not part of its natural environment. In preferred embodiments, a nucleic acid or protein is said to be purified, for example, if it gives rise to essentially one band in an electrophoretic gel or blot.

The term “isolated”, when used in reference to a DNA sequence, refers to a DNA sequence that has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (See e.g., Dynan and Tijan, Nature 316:774-78, 1985). The term “an isolated DNA sequence” is alternatively referred to as “a cloned DNA sequence”.

The term “isolated,” when used in reference to a protein, refers to a protein that is found in a condition other than its native environment. In a preferred form, the isolated protein is substantially free of other proteins, particularly other homologous proteins. An isolated protein is more than about 10% pure, preferably more than about 20% pure, and even more preferably more than about 30% pure, as determined by SDS-PAGE. Further aspects of the invention encompass the protein in a highly purified form (i.e., more than about 40% pure, more than about 60% pure, more than about 80% pure, more than about 90% pure, more than about 95% pure, more than about 97% pure, and even more than about 99% pure), as determined by SDS-PAGE.

The following cassette mutagenesis method may be used to facilitate the construction of the enzyme variants of the present invention, although other methods may be used. First, as described herein, a naturally-occurring gene encoding the enzyme is obtained and sequenced in whole or in part. Then, the sequence is scanned for a point at which it is desired to make a mutation (deletion, insertion or substitution) of one or more amino acids in the encoded enzyme. The sequences flanking this point are evaluated for the presence of restriction sites for replacing a short segment of the gene with an oligonucleotide pool which when expressed will encode various mutants. Such restriction sites are preferably unique sites within the protein gene so as to facilitate the replacement of the gene segment. However, any convenient restriction site that is not overly redundant in the enzyme gene may be used, provided the gene fragments generated by restriction digestion can be reassembled in proper sequence. If restriction sites are not present at locations within a convenient distance from the selected point (from 10 to 15 nucleotides), such sites are generated by substituting nucleotides in the gene in such a fashion that neither the reading frame nor the amino acids encoded are changed in the final construction. Mutation of the gene in order to change its sequence to conform to the desired sequence is accomplished by M13 primer extension in accord with generally known methods. The task of locating suitable flanking regions and evaluating the needed changes to arrive at two convenient restriction site sequences is made routine by the redundancy of the genetic code, a restriction enzyme map of the gene and the large number of different restriction enzymes. Note that if a convenient flanking restriction site is available, the above method need be used only in connection with the flanking region that does not contain a site.

Once the naturally-occurring DNA and/or synthetic DNA is cloned, the restriction sites flanking the positions to be mutated are digested with the cognate restriction enzymes and a plurality of end termini-complementary oligonucleotide cassettes are ligated into the gene. The mutagenesis is simplified by this method because all of the oligonucleotides can be synthesized so as to have the same restriction sites, and no synthetic linkers are necessary to create the restriction sites.

As used herein, “corresponding to,” refers to a residue at the enumerated position in a protein or peptide, or a residue that is analogous, homologous, or equivalent to an enumerated residue in a protein or peptide.

As used herein, “corresponding region,” generally refers to an analogous position along related proteins or a parent protein.

As used herein, the term, “combinatorial mutagenesis” refers to methods in which libraries of variants of a starting sequence are generated. In these libraries, the variants contain one or several mutations chosen from a predefined set of mutations. In addition, the methods provide means to introduce random mutations, which were not members of the predefined set of mutations. In some embodiments, the methods include those set forth in U.S. application Ser. No. 09/699,250, filed Oct. 26, 2000, hereby incorporated by reference. In alternative embodiments, combinatorial mutagenesis methods encompass commercially available kits (e.g., QUIKCHANGE® Multisite, Stratagene, San Diego, Calif.).

As used herein, the term “library of mutants” refers to a population of cells which are identical in most of their genome but include different homologues of one or more genes. Such libraries can be used, for example, to identify genes or operons with improved traits.

As used herein, the terms “starting gene” and “parent gene” refer to a gene of interest that encodes a protein of interest that is to be improved and/or changed using the present invention.

As used herein, the terms “multiple sequence alignment” and “MSA” refer to the sequences of multiple homologs of a starting gene that are aligned using an algorithm (e.g., Clustal W).

As used herein, the terms “consensus sequence” and “canonical sequence” refer to an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in the MSA.

As used herein, the term “consensus mutation” refers to a difference in the sequence of a starting gene and a consensus sequence. Consensus mutations are identified by comparing the sequences of the starting gene and the consensus sequence obtained from a MSA. In some embodiments, consensus mutations are introduced into the starting gene such that it becomes more similar to the consensus sequence. Consensus mutations also include amino acid changes that change an amino acid in a starting gene to an amino acid that is more frequently found in an MSA at that position relative to the frequency of that amino acid in the starting gene. Thus, the term consensus mutation comprises all single amino acid changes that replace an amino acid of the starting gene with an amino acid that is more abundant than the amino acid in the MSA.

The terms “modified sequence” and “modified genes” are used interchangeably herein to refer to a sequence that includes a deletion, insertion or interruption of naturally occurring nucleic acid sequence. In some preferred embodiments, the expression product of the modified sequence is a truncated protein (e.g., if the modification is a deletion or interruption of the sequence). In some particularly preferred embodiments, the truncated protein retains biological activity. In alternative embodiments, the expression product of the modified sequence is an elongated protein (e.g., modifications comprising an insertion into the nucleic acid sequence). In some embodiments, an insertion leads to a truncated protein (e.g., when the insertion results in the formation of a stop codon). Thus, an insertion may result in either a truncated protein or an elongated protein as an expression product.

As used herein, the terms “mutant sequence” and “mutant gene” are used interchangeably and refer to a sequence that has an alteration in at least one codon occurring in a host cell's wild-type sequence. The expression product of the mutant sequence is a protein with an altered amino acid sequence relative to the wild-type. The expression product may have an altered functional capacity (e.g., enhanced enzymatic activity).

The terms “mutagenic primer” or “mutagenic oligonucleotide” (used interchangeably herein) are intended to refer to oligonucleotide compositions which correspond to a portion of the template sequence and which are capable of hybridizing thereto. With respect to mutagenic primers, the primer will not precisely match the template nucleic acid, the mismatch or mismatches in the primer being used to introduce the desired mutation into the nucleic acid library. As used herein, “non-mutagenic primer” or “non-mutagenic oligonucleotide” refers to oligonucleotide compositions that match precisely to the template nucleic acid. In one embodiment of the invention, only mutagenic primers are used. In another preferred embodiment of the invention, the primers are designed so that for at least one region at which a mutagenic primer has been included, there is also non-mutagenic primer included in the oligonucleotide mixture. By adding a mixture of mutagenic primers and non-mutagenic primers corresponding to at least one of the mutagenic primers, it is possible to produce a resulting nucleic acid library in which a variety of combinatorial mutational patterns are presented. For example, if it is desired that some of the members of the mutant nucleic acid library retain their parent sequence at certain positions while other members are mutant at such sites, the non-mutagenic primers provide the ability to obtain a specific level of non-mutant members within the nucleic acid library for a given residue. The methods of the invention employ mutagenic and non-mutagenic oligonucleotides which are generally between 10-50 bases in length, more preferably about 15-45 bases in length. However, it may be necessary to use primers that are either shorter than 10 bases or longer than 50 bases to obtain the mutagenesis result desired. With respect to corresponding mutagenic and non-mutagenic primers, it is not necessary that the corresponding oligonucleotides be of identical length, but only that there is overlap in the region corresponding to the mutation to be added.

Primers may be added in a pre-defined ratio according to the present invention. For example, if it is desired that the resulting library have a significant level of a certain specific mutation and a lesser amount of a different mutation at the same or different site, by adjusting the amount of primer added, it is possible to produce the desired biased library. Alternatively, by adding lesser or greater amounts of non-mutagenic primers, it is possible to adjust the frequency with which the corresponding mutation(s) are produced in the mutant nucleic acid library.

The terms “wild-type sequence,” or “wild-type gene” are used interchangeably herein, to refer to a sequence that is native or naturally occurring in a host cell. In some embodiments, the wild-type sequence refers to a sequence of interest that is the starting point of a protein-engineering project. The wild-type sequence may encode either a homologous or heterologous protein. A homologous protein is one the host cell would produce without intervention. A heterologous protein is one that the host cell would not produce but for the intervention.

As used herein, the term “cleaning composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

Unless otherwise noted, all component or composition levels are in reference to the active level of that component or composition, and are exclusive of impurities, for example, residual solvents or by-products, which may be present in commercially available sources.

Enzyme components weights are based on total active protein. All percentages and ratios are calculated by weight unless otherwise indicated. All percentages and ratios are calculated based on the total composition unless otherwise indicated.

The term “cleaning activity” refers to the cleaning performance achieved by the protease under conditions prevailing during the proteolytic, hydrolyzing, cleaning or other process of the invention. In some embodiments, cleaning performance is determined by the application of various cleaning assays concerning enzyme sensitive stains, for example grass, blood, milk, or egg protein as determined by various chromatographic, spectrophotometric or other quantitative methodologies after subjection of the stains to standard wash conditions. Exemplary assays include, but are not limited to those described in WO 99/34011, and U.S. Pat. No. 6,605,458 (both of which are herein incorporated by reference), as well as those methods included in the Examples.

The term “cleaning effective amount” of a protease refers to the quantity of protease described hereinbefore that achieves a desired level of enzymatic activity in a specific cleaning composition. Such effective amounts are readily ascertained by one of ordinary skill in the art and are based on many factors, such as the particular protease used, the cleaning application, the specific composition of the cleaning composition, and whether a liquid or dry (e.g., granular, bar) composition is required, etc.

The term “cleaning adjunct materials” as used herein, means any liquid, solid or gaseous material selected for the particular type of cleaning composition desired and the form of the product (e.g., liquid, granule, powder, bar, paste, spray, tablet, gel; or foam composition), which materials are also preferably compatible with the protease enzyme used in the composition. In some embodiments, granular compositions are in “compact” form, while in other embodiments, the liquid compositions are in a “concentrated” form.

As used herein, a “low detergent concentration” system includes detergents where less than about 800 ppm of detergent components are present in the wash water. Japanese detergents are typically considered low detergent concentration systems, as they usually have approximately 667 ppm of detergent components present in the wash water.

As used herein, a “medium detergent concentration” systems includes detergents wherein between about 800 ppm and about 2000 ppm of detergent components are present in the wash water. North American detergents are generally considered to be medium detergent concentration systems as they have usually approximately 975 ppm of detergent components present in the wash water. Brazilian detergents typically have approximately 1500 ppm of detergent components present in the wash water.

As used herein, “high detergent concentration” systems includes detergents wherein greater than about 2000 ppm of detergent components are present in the wash water. European detergents are generally considered to be high detergent concentration systems as they have approximately 3000-8000 ppm of detergent components in the wash water.

As used herein, “fabric cleaning compositions” include hand and machine laundry detergent compositions including laundry additive compositions and compositions suitable for use in the soaking and/or pretreatment of stained fabrics (e.g., clothes, linens, and other textile materials).

As used herein, “non-fabric cleaning compositions” include non-textile (i.e., fabric) surface cleaning compositions, including but not limited to dishwashing detergent compositions, oral cleaning compositions, denture cleaning compositions, and personal cleansing compositions.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically 17-35% by weight of the total composition. In contrast, in compact compositions, the filler salt is present in amounts not exceeding 15% of the total composition. In some embodiments, the filler salt is present in amounts that do not exceed 10%, or more preferably, 5%, by weight of the composition. In some embodiments, the inorganic filler salts are selected from the alkali and alkaline-earth-metal salts of sulfates and chlorides. A preferred filler salt is sodium sulfate.

The term “endogenous” when used in reference to a protein (e.g., enzyme) indicates that it has been expressed from a native gene of a host cell or organism of interest.

The term “heterologous” when used herein in reference to a protein (e.g., enzyme) indicates that it has been expressed from a foreign gene introduced into a host cell or organism of interest.

As used herein the terms “serine protease-free,” “relative absence of serine protease,” and “essentially devoid of serine protease,” refer to a composition that contains little to no measurable serine protease (e.g., protein or activity at or below the level of detection). In some embodiments, the serine protease content of the composition is less than 0.050 U/ml, preferably less than 0.025 U/ml, more preferably less than 0.005 U/ml, and most preferably less than 0.0025 U/ml (e.g., as measured in an AAPF assay). In some embodiments, the composition comprises a neutral metalloprotease.

As used herein, the term “serine protease-free background” and the like refer to the production of a protein of interest by an organism (e.g., serine protease deficient production strain) that expresses little to no measurable serine protease (e.g., protein or activity). In some preferred embodiments, the organism is modified such that the gene(s) encoding at least one serine protease have been deleted or mutated such that the organism is no longer capable of producing the serine protease(s). In some embodiments, the serine protease deficient production strain expresses less than about 1%, preferably less than about 0.5%, more preferably less than about 0.1% and most preferably less than about 0.05% of the serine protease activity of the corresponding wild type strain (or parental strain not comprising a serine protease gene deletion or inactivation).

### DETAILED DESCRIPTION OF THE PRESENT INVENTION

Neutral metalloendopeptidases (i.e., neutral metalloproteases) (EC 3.4.24.4) belong to a protease class that has an absolute requirement for zinc ions for catalytic activity. These enzymes are optimally active at neutral pH and are in the 30 to 40 kDa size range. Neutral metalloproteases bind between two and four calcium ions that contribute to the structural stability of the protein. The bound metal ion at the active site of metalloproteases is an essential feature that allows the activation of a water molecule. The water molecule then functions as the nucleophile and cleaves the carbonyl group of the peptide bond.

The present invention provides methods and compositions comprising at least one neutral metalloprotease enzyme in the relative absence of serine protease enzyme contaminants. In some embodiments, the neutral metalloprotease finds use in cleaning and other applications. In some particularly preferred embodiments, the present invention provides methods and compositions comprising Bacillus strains engineered to be deficient in multiple serine proteases, and their use in production of recombinant neutral metalloprotease(s).

As described herein, an integrating plasmid for the expression of NprE was made and transformed into Bacillus host strains having deletions or inactivation of one or more enzyme genes. The integrating plasmid was then transformed into the 2-delete host strain creating EL534 and EL535 (e.g., Example 7). The chromosomal DNA of EL534 was then transformed into the 5-delete host strain (e.g., Example 10) and the 8-delete host strain (e.g., Example 12). Fermentation tanks were run for the 2-delete, 5-delete, and 8-delete strains to assess residual serine protease contamination. Fermentation samples from the three host strains were analyzed on an SDS-PAGE gel, by AAPF assay, and by N-terminal sequencing of the protein bands of the host cell supernatants that ranged in size from 20-30 kDa and 100 kDa. In this way, Vpr was identified as the putative contaminating serine protease. Chromosomal DNA from strain EL534 was transformed into the 3-delete, 4-delete, and 6-delete strains. Analysis of the fermentation samples for the 3-delete (EL552), 4-delete (EL553), and 6-delete (EL547) strains indicated that EL547 also provides a serine protease-free background. As such in some preferred embodiments of the present invention, the 6-delete strain and the 8-delete strain are employed for production of recombinant neutral metalloproteases.

### Detailed Description of Cleaning and Detergent Formulations of the Present Invention

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated.

In the exemplified detergent compositions, the enzymes levels are expressed by pure enzyme by weight of the total composition and unless otherwise specified, the detergent ingredients are expressed by weight of the total compositions.

### Cleaning Compositions Comprising Neutral Metalloprotease

The neutral metalloproteases of the present invention are useful in formulating various detergent compositions. The cleaning composition of the present invention may be advantageously employed for example, in laundry applications, hard surface cleaning, automatic dishwashing applications, as well as cosmetic applications such as dentures, teeth, hair and skin. However, due to the unique advantages of increased effectiveness in lower temperature solutions and the superior color-safety profile, the enzymes of the present invention are ideally suited for laundry applications such as the bleaching of fabrics. Furthermore, the enzymes of the present invention find use in both granular and liquid compositions.

The enzymes of the present invention also find use in cleaning additive products. A cleaning additive product including at least one enzyme of the present invention is ideally suited for inclusion in a wash process when additional bleaching effectiveness is desired. Such instances include, but are not limited to low temperature solution cleaning applications. The additive product may be, in its simplest form, one or more neutral metalloprotease enzyme as provided by the present invention. In some embodiments, the additive is packaged in dosage form for addition to a cleaning process where a source of peroxygen is employed and increased bleaching effectiveness is desired. In some embodiments, the single dosage form comprises a pill, tablet, gelcap or other single dosage unit including pre-measured powders and/or liquids. In some embodiments, filler and/or carrier material(s) are included, in order to increase the volume of such composition. Suitable filler or carrier materials include, but are not limited to, various salts of sulfate, carbonate and silicate as well as talc, clay and the like. In some embodiments filler and/or carrier materials for liquid compositions include water and/or low molecular weight primary and secondary alcohols including polyols and diols. Examples of such alcohols include, but are not limited to, methanol, ethanol, propanol and isopropanol. In some embodiments, the compositions comprise from about 5% to about 90% of such materials. In additional embodiments, acidic fillers are used to reduce the pH of the composition. In some alternative embodiments the cleaning additive includes at least one activated peroxygen source as described below and/or adjunct ingredients as more fully described below.

The cleaning compositions and cleaning additives of the present invention require an effective amount of neutral metalloprotease enzyme as provided in the present invention. In some embodiments, the required level of enzyme is achieved by the addition of one or more species of neutral metalloprotease provided by the present invention. Typically, the cleaning compositions of the present invention comprise at least 0.0001 weight percent, from about 0.0001 to about 1, from about 0.001 to about 0.5, or even from about 0.01 to about 0.1 weight percent of at least one neutral metalloprotease provided by the present invention.

In some preferred embodiments, the cleaning compositions provided herein are typically formulated such that, during use in aqueous cleaning operations, the wash water has a pH of from about 5.0 to about 11.5, or in alternative embodiments, even from about 6.0 to about 10.5. In some preferred embodiments, liquid product formulations are typically formulated to have a neat pH from about 3.0 to about 9.0, while in some alternative embodiments the formulation has a neat pH from about 3 to about 5. In some preferred embodiments, granular laundry products are typically formulated to have a pH from about 8 to about 11. Techniques for controlling pH at recommended usage levels include the use of buffers, alkalis, acids, etc., and are well known to those skilled in the art.

In some particularly preferred embodiments, when at least one neutral metalloprotease is employed in a granular composition or liquid, the neutral metalloprotease is in the form of an encapsulated particle to protect the enzyme from other components of the granular composition during storage. In addition, encapsulation also provides a means of controlling the availability of the neutral metalloprotease(s) during the cleaning process and may enhance performance of the neutral metalloprotease(s). It is contemplated that the encapsulated neutral metalloproteases of the present invention will find use in various settings. It is also intended that the neutral metalloprotease be encapsulated using any suitable encapsulating material(s) and method(s) known in the art.

In some preferred embodiments, the encapsulating material typically encapsulates at least part of the neutral metalloprotease catalyst. In some embodiments, the encapsulating material is water-soluble and/or water-dispersible. In some additional embodiments, the encapsulating material has a glass transition temperature (Tg) of 0° C. or higher (See e.g., WO 97/11151, particularly from page 6, line 25 to page 7, line 2, for more information regarding glass transition temperatures).

In some embodiments, the encapsulating material is selected from carbohydrates, natural or synthetic gums, chitin and chitosan, cellulose and cellulose derivatives, silicates, phosphates, borates, polyvinyl alcohol, polyethylene glycol, paraffin waxes and combinations thereof. In some embodiments in which the encapsulating material is a carbohydrate, it is selected from monosaccharides, oligosaccharides, polysaccharides, and combinations thereof. In some preferred embodiments, the encapsulating material is a starch (See e.g., EP 0 922 499; U.S. Pat. No. 4,977,252. U.S. Pat. No. 5,354,559, and U.S. Pat. No. 5,935,826, for descriptions of some exemplary suitable starches).

In additional embodiments, the encapsulating material comprises a microsphere made from plastic(e.g., thermoplastics, acrylonitrile, methacrylonitrile, polyacrylonitrile, polymethacrylonitrile and mixtures thereof; commercially available microspheres that find use include, but are not limited to EXPANCEL® [Casco Products, Stockholm, Sweden], PM 6545, PM 6550, PM 7220, PM 7228, EXTENDOSPHERES®, and Q-CEL® [PQ Corp., Valley Forge, Pa.], LUXSIL® and SPHERICELl® [Potters Industries, Inc., Carlstadt, N.J. and Valley Forge, Pa.]).

### Processes of Making and Using of Applicants' Cleaning Composition

In some preferred embodiments compositions of the present invention are formulated into any suitable form and prepared by any process chosen by the formulator (See e.g., U.S. Pat. No. 5,879,584, U.S. Pat. No. 5,691,297, U.S. Pat. No. 5,574,005, U.S. Pat. No. 5,569,645, U.S. Pat. No. 5,565,422, U.S. Pat. No. 5,516,448, U.S. Pat. No. 5,489,392, and U.S. Pat. No. 5,486,303, for some non-limiting examples). In some embodiments in which a low pH cleaning composition is desired, the pH of such composition is adjusted via the addition of an acidic material such as HCl.

### Adjunct Materials

While not essential for the purposes of the present invention, in some embodiments, the non-limiting list of adjuncts described herein are suitable for use in the cleaning compositions of the present invention. Indeed, in some embodiments, adjuncts are incorporated into the cleaning compositions of the present invention. In some embodiments, adjunct materials assist and/or enhance cleaning performance, treat the substrate to be cleaned, and/or modify the aesthetics of the cleaning composition (e.g., perfumes, colorants, dyes, etc.). It is understood that such adjuncts are in addition to the neutral metalloproteases of the present invention. The precise nature of these additional components, and levels of incorporation thereof, depends on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to those provided explicitly herein, additional examples are known in the art (See e.g., U.S. Pat. Nos. 5,576,282, 6,306,812 B1 and 6,326,348 B1). In some embodiments, the aforementioned adjunct ingredients constitute the balance of the cleaning compositions of the present invention.

Surfactants—In some embodiments, the cleaning compositions of the present invention comprise at least one surfactant or surfactant system, wherein the surfactant is selected from nonionic surfactants, anionic surfactants, cationic surfactants, ampholytic surfactants, zwitterionic surfactants, semi-polar nonionic surfactants, and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions the acidic contents.

In some embodiments, the surfactant is present at a level of from about 0.1% to about 60%, while in alternative embodiments, the level is from about 1% to about 50%, while in still further embodiments, the level is from about 5% to about 40%, by weight of the cleaning composition.

Builders—In some embodiments, the cleaning compositions of the present invention comprise one or more detergent builders or builder systems. In some embodiments incorporating at least one builder, the cleaning compositions comprise at least about 1%, from about 3% to about 60% or even from about 5% to about 40% builder by weight of the cleaning composition.

Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present invention.

Chelating Agents—In some embodiments, the cleaning compositions of the present invention contain at least one chelating agent. Suitable chelating agents include, but are not limited to copper, iron and/or manganese chelating agents and mixtures thereof. In embodiments in which at least one chelating agent is used, the cleaning compositions of the present invention comprise from about 0.1% to about 15% or even from about 3.0% to about 10% chelating agent by weight of the subject cleaning composition.

Deposition Aid—In some embodiments, the cleaning compositions of the present invention include at least one deposition aid. Suitable deposition aids include, but are not limited to polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

Dye Transfer Inhibiting Agents—In some embodiments, the cleaning compositions of the present invention include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof.

In embodiments in which at least one dye transfer inhibiting agent is used, the cleaning compositions of the present invention comprise from about 0.0001% to about 10%, from about 0.01% to about 5%, or even from about 0.1% to about 3% by weight of the cleaning composition.

Dispersants—In some embodiments, the cleaning compositions of the present invention contains at least one dispersants. Suitable water-soluble organic materials include, but are not limited to the homo- or co-polymeric acids or their salts, in which the polycarboxylic acid comprises at least two carboxyl radicals separated from each other by not more than two carbon atoms.

Enzymes—In some embodiments, the cleaning compositions of the present invention comprise one or more detergent enzymes, which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. In some embodiments, a combination of enzymes is used (i.e., a “cocktail”) comprising conventional applicable enzymes like protease, lipase, cutinase and/or cellulase in conjunction with amylase is used.

Enzyme Stabilizers—In some embodiments of the present invention, the enzymes used in the detergent formulations of the present invention are stabilized. It is contemplated that various techniques for enzyme stabilization will find use in the present invention. For example, in some embodiments, the enzymes employed herein are stabilized by the presence of water-soluble sources of zinc (II), calcium (II) and/or magnesium (II) ions in the finished compositions that provide such ions to the enzymes, as well as. other metal ions (e.g., barium (II), scandium (II), iron (II), manganese (II), aluminum (III), Tin (II), cobalt (II), copper (II), Nickel (II), and oxovanadium (IV)).

Catalytic Metal Complexes—In some embodiments, the cleaning compositions of the present invention contain one or more catalytic metal complexes. In some embodiments, a metal-containing bleach catalyst finds use. In some preferred embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See e.g., U.S. Pat. No. 4,430,243).

In some embodiments, the cleaning compositions of the present invention are catalyzed by means of a manganese compound. Such compounds and levels of use are well known in the art (See e.g., U.S. Pat. No. 5,576,282).

In additional embodiments, cobalt bleach catalysts find use in the cleaning compositions of the present invention. Various cobalt bleach catalysts are known in the art (See e.g., U.S. Pat. No. 5,597,936, and U.S. Pat. No. 5,595,967). Such cobalt catalysts are readily prepared by known procedures (See e.g., U.S. Pat. No. 5,597,936, and U.S. Pat. No. 5,595,967).

In additional embodiments, the cleaning compositions of the present invention include a transition metal complex of a macropolycyclic rigid ligand (“MRL”). As a practical matter, and not by way of limitation, in some embodiments, the compositions and cleaning processes provided by the present invention are adjusted to provide on the order of at least one part per hundred million of the active MRL species in the aqueous washing medium, and in some preferred embodiments, provide from about 0.005 ppm to about 25 ppm, more preferably from about 0.05 ppm to about 10 ppm, and most preferably from about 0.1 ppm to about 5 ppm, of the MRL in the wash liquor.

Preferred transition-metals in the instant transition-metal bleach catalyst include, but are not limited to manganese, iron and chromium. Preferred MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See e.g., WO 00/32601, and U.S. Pat. No. 6,225,464).

### Processes of Making and Using Cleaning Compositions

The cleaning compositions of the present invention are formulated into any suitable form and prepared by any suitable process chosen by the formulator, (See e.g., U.S. Pat. No. 5,879,584, U.S. Pat. No. 5,691,297, U.S. Pat. No. 5,574,005, U.S. Pat. No. 5,569,645, U.S. Pat. No. 5,565,422, U.S. Pat. No. 5,516,448, U.S. Pat. No. 5,489,392, U.S. Pat. No. 5,486,303, U.S. Pat. No. 4,515,705, U.S. Pat. No. 4,537,706, U.S. Pat. No. 4,515,707, U.S. Pat. No. 4,550,862, U.S. Pat. No. 4,561,998, U.S. Pat. No. 4,597,898, U.S. Pat. No. 4,968,451, U.S. Pat. No. 5,565,145, U.S. Pat. No. 5,929,022, U.S. Pat. No. 6,294,514, and U.S. Pat. No. 6,376,445, all of which are incorporated herein by reference for some non-limiting examples).

### Method of Use

In preferred embodiments, the cleaning compositions of the present invention find use in cleaning surfaces and/or fabrics. In some embodiments, at least a portion of the surface and/or fabric is contacted with at least one embodiment of the cleaning compositions of the present invention, in neat form or diluted in a wash liquor, and then the surface and/or fabric is optionally washed and/or rinsed. For purposes of the present invention, “washing” includes, but is not limited to, scrubbing, and mechanical agitation. In some embodiments, the fabric comprises any fabric capable of being laundered in normal consumer use conditions. In preferred embodiments, the cleaning compositions of the present invention are used at concentrations of from about 500 ppm to about 15,000 ppm in solution. In some embodiments in which the wash solvent is water, the water temperature typically ranges from about 5° C. to about 90° C. In some preferred embodiments for fabric cleaning, the water to fabric mass ratio is typically from about 1:1 to about 30:1.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa and AA (amino acid); by (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); nm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD (optical density; PAGE (polyacrylamide gel electrophoresis); EtOH (ethanol); PBS (phosphate buffered saline [150 mM NaCl, 10 mM sodium phosphate buffer, pH 7.2]); LAS (lauryl sodium sulfonate); SDS (sodium dodecyl sulfate); Tris (tris(hydroxymethyl)aminomethane); TAED (N,N,N′N′-tetraacetylethylenediamine); BES (polyesstersulfone); MES (2-morpholinoethanesulfonic acid, monohydrate; f.w. 195.24; Sigma # M-3671); CaCl₂ (calcium chloride, anhydrous; f.w. 110.99; Sigma # C-4901); DMF (N,N-dimethylformamide, f.w. 73.09, d=0.95); Abz-AGLA-Nba (2-aminobenzoyl-L-alanyl-glycyl-L-leucyl-L-alamino-4-nitrobenzylamide, f.w. 583.65; Bachem #H-6675, VWR catalog #100040-598); SBG1% (“Super Broth with Glucose”; 6 g Soytone [Difco], 3 g yeast extract, 6 g NaCl, 6 g glucose); the pH was adjusted to 7.1 with NaOH prior to sterilization using methods known in the art; w/v (weight to volume); v/v (volume to volume); Npr and npr (neutral metalloprotease); SEQUEST® (SEQUEST database search program, University of Washington); Npr and npr (neutral metalloprotease gene); NprE and nprE (B. amyloliquefaciens neutral metalloprotease); PMN (purified MULTIFECT® metalloprotease); MS (mass spectroscopy); and SRI (Stain Removal Index).

The following abbreviations apply to companies whose products or services may have been referred to in the experimental examples: TIGR (The Institute for Genomic Research, Rockville, Md.); AATCC (American Association of Textile and Coloring Chemists); Amersham (Amersham Life Science, Inc. Arlington Heights, Ill.); Corning (Corning International, Corning, N.Y.); ICN (ICN Pharmaceuticals, Inc., Costa Mesa, Calif.); Pierce (Pierce Biotechnology, Rockford, Ill.); Equest (Equest, Warwick International Group, Inc., Flintshire, UK); EMPA (Eidgenossische Material Prufungs and Versuch Anstalt, St. Gallen, Switzerland); CFT (Center for Test Materials, Vlaardingen, The Netherlands); Amicon (Amicon, Inc., Beverly, Mass.); ATCC (American Type Culture Collection, Manassas, Va.); Becton Dickinson (Becton Dickinson Labware, Lincoln Park, N.J.); Perkin-Elmer (Perkin-Elmer, Wellesley, Mass.); Rainin (Rainin Instrument, LLC, Woburn, Mass.); Eppendorf (Eppendorf AG, Hamburg, Germany); Waters (Waters, Inc., Milford, Mass.); Geneart (Geneart GmbH, Regensburg, Germany); Perseptive Biosystems (Perseptive Biosystems, Ramsey, Minn.); Molecular Probes (Molecular Probes, Eugene, Oreg.); BioRad (BioRad, Richmond, Calif.); Clontech (CLONTECH Laboratories, Palo Alto, Calif.); Cargill (Cargill, Inc., Minneapolis, Minn.); Difco (Difco Laboratories, Detroit, Mich.); GIBCO BRL or Gibco BRL (Life Technologies, Inc., Gaithersburg, Md.); New Brunswick (New Brunswick Scientific Company, Inc., Edison, N.J.); Thermoelectron (Thermoelectron Corp., Waltham, Mass.); BMG (BMG Labtech, GmbH, Offenburg, Germany); Greiner (Greiner Bio-One, Kremsmuenster, Austria); Novagen (Novagen, Inc., Madison, Wis.); Novex (Novex, San Diego, Calif.); Finnzymes (Finnzymes OY, Finland) Qiagen (Qiagen, Inc., Valencia, Calif.); Invitrogen (Invitrogen Corp., Carlsbad, Calif.); Sigma (Sigma Chemical Co., St. Louis, Mo.); DuPont Instruments (Asheville, N.Y.); Global Medical Instrumentation or GMI (Global Medical Instrumentation; Ramsey, Minn.); MJ Research (MJ Research, Waltham, Mass.); Infors (Infors AG, Bottmingen, Switzerland); Stratagene (Stratagene Cloning Systems, La Jolla, Calif.); Roche (Hoffmann La Roche, Inc., Nutley, N.J.); Agilent (Agilent Technologies, Palo Alto, Calif.); S-Matrix (S-Matrix Corp., Eureka, Calif.); US Testing (United States Testing Co., Hoboken, N.Y.); West Coast Analytical Services (West Coast Analytical Services, Inc., Santa Fe Springs, Calif.); Ion Beam Analysis Laboratory (Ion Bean Analysis Laboratory, The University of Surrey Ion Beam Centre (Guildford, UK); TOM (Terg-o-Meter); BMI (blood, milk, ink); BaChem (BaChem AG, Bubendorf, Switzerland); Molecular Devices (Molecular Devices, Inc., Sunnyvale, Calif.); Corning (Corning International, Corning, N.Y.); MicroCal (Microcal, Inc., Northhampton, Mass.); Chemical Computing (Chemical Computing Corp., Montreal, Canada); NCBI (National Center for Biotechnology Information); Argo Bioanalytica (Argo Bioanalytica. Inc, New Jersey); Vydac (Grace Vydac, Hesperia, Calif.); Minolta (Konica Minolta, Ramsey, N.J.); and Zeiss (Carl Zeiss, Inc., Thornwood, N.Y.).

### Example 1

### Assays and Detergents

The following assays were used in the examples described below or other analyses of recombinant proteases. Any deviations from the protocols provided below are indicated in the examples. In these experiments, a spectrophotometer was used to measure the absorbance of the products formed after the completion of the reactions. A reflectometer was used to measure the reflectance of the swatches.

### A. Protein Content Determination

1. BCA (Bicinchoninic Acid) Assay for Protein Content Determination in 96-Well Microtiter Plates (MTPs)

In these assays, BCA (Pierce) assay was used to determine the protein concentration in protease samples on MTP scale. In this assay system, the chemical and reagent solutions used were: BCA protein assay reagent, and Pierce Dilution buffer (50 mM MES, pH 6.5, 2 mM CaCl₂, 0.005% TWEEN®-80). The equipment used was a SpectraMAX (type 340) MTP reader. The MTPs were obtained from Costar (type 9017).

In the test, 200 μl BCA Reagent was pipetted into each well, followed by 20 μl diluted protein. After thorough mixing, the MTPs were incubated for 30 minutes at 37° C. Possible air bubbles were removed, and the optical density (OD) of the solution within the wells was read at 562 nm. To determine the protein concentration, the background reading was subtracted form the sample readings. The OD₅₆₂ was plotted for against protein standards (purified protease), to produce a standard curve. The protein concentration of the samples was extrapolated from the standard curve.

2. Bradford Assay for Protein Content Determination in 96-Well Microtiter Plates (MTPs)

In these assays, the Bradford dye reagent (Quick Start) assay was used to determine the protein concentration in protease samples on MTP scale.

In this assay system, the chemical and reagent solutions used were: Quick Start Bradford Dye Reagent (BIO-RAD Catalog No. 500-0205), Dilution buffer (10 mM NaCl, 0.1 mM CaCl2, 0.005% TWEEN®-80). The equipment used was a Biomek FX Robot (Beckman) and a SpectraMAX (type 340) MTP reader. The MTPs were from Costar (type 9017).

In the test, 200 μl Bradford Dye Reagent was pipetted into each well, followed by 15 μl dilution buffer. Finally 10 μl of filtered culture broth were added to the wells. After thorough mixing, the MTPs were incubated for at least 10 minutes at room temperature. Possible air bubbles were blown away and the ODs of the wells were read at 595 nm. To determine the protein concentration, the background reading (i.e., from uninoculated wells) was subtracted form the sample readings. The obtained OD₅₉₅ values provide a relative measure of the protein content in the samples.

### B. Protease Assays

1) Azo-Casein Assay:

The azo-casein endpoint assay was used to assess the amount of proteolysis that occurred under certain conditions. In these assays, 75 uL of enzyme were incubated with excess calcium or zinc or both ions added to 250 μl of 1% (w/v) azo-casein (Sigma). The reaction proceeded at 30° C. for 15 minutes, after which 10% (w/v) trichloroacetic acid (TCA) was added to stop the reaction. The precipitated protein and the unreacted azo-casein were removed by centrifugation for 10 minutes at 14,000 rpm. The color of the azo-group was developed by addition of 750 μL 1 M sodium hydroxide. The development of the color proceeded for 5 minutes, after which the reaction was stopped and the absorbance was measured at 440 nm.

2) Succinylated-Casein Assay:

The activity of the neutral metalloprotease (NprE) was determined using the QuantiCleave Protease Assay Kit™ (Pierce). This assay is based on the digestion of succinylated-casein by the enzyme. The primary amino groups formed are then reacted with trinitrobenzene sulfonic acid (TNBSA) and form a colored complex that has maximum absorbance at 450 nm. The assay is performed in 96-well microtiter format. The assay requires a 15-minute incubation with the succinylated casein and a 15-minute reaction with the TNBSA. During both incubations, the samples are placed on a shaker. TPCK-trypsin (Pierce) is the general standard used for overall protease activity determinations. However, optimum conditions for activity for specific proteases require the use of the protease of interest. In the case of the assays performed in these experiments, both trypsin and the protease of interest were used, in order to calibrate the assay. The accuracy of the assay requires that the standard dilutions made of 0.5 mg/mL trypsin always result in absorbance values (at 450 nm) below 0.5.

Every sample was measured relative to a control containing no casein. The reported change in absorbance (ΔAbs at 450 nm) accounts for the interference from the amino groups of casein. Further, any possible interference from primary amino groups in the buffer and/or other components of the detergent was/were also corrected for in this manner. The activity of all samples was determined relative to detergent with no added neutral metalloprotease, as well as for enzyme incubated in BupH™ borate buffer supplied with the kit, for the same length of time and at the same temperature.

This test is an end-point assay, in which 50 mM borate buffer, pH 8.5, was used at 32° C. The protease assays were typically performed in duplicate. In most experiments to determine stability measurements, the protein and detergent were diluted using the above-mentioned buffer by 1:1000, although in some experiments dilutions of were also 1:500 or 1: 200, in order to obtain readings where the absorbance of the blanks was less than 0.5. The microtiter spectrophotometer used in these experiments was a SpectraMax250® (Molecular Devices) and all assays were conducted in medium protein-binding 96-well plates (Corning).

The results for the standards protein samples (e.g., trypsin and purified metalloprotease) obtained in these assays indicated that there was a non-linear response (a linear scale may be adequate only in a narrow assay range). Hence, the curve was fitted to a quadratic function where f=y₀+ax²+bx; f is fit to y (SigmaPlot® v. 9; SPSS, Inc.). Thus, if a linear equation was used to quantitate the amount of protein, inaccurate data were obtained; the quadratic equation was required in order to obtain accurate results. It is noted that the manufacturer's (Pierce) kit insert indicates that the results may be fitted with “x” being a log scale.

3. Dimethylcasein (DMC) Hydrolysis Assay

In this assay system, the chemicals and reagent solutions used were:

| | |
|---|---|
| Dimethylcasein (DMC) | Sigma C-9801 |
| TWEEN ®-80 | Sigma P-8074 |
| PIPES buffer (free acid) | Sigma P-1851; 15.1 g dissolved in about |
| brought | 960 ml water; pH adjusted to 6.0 with 4N |
| of PIPES and | NaOH, 1 ml of 5% TWEEN ®-80 added and |
| respectively. | the volume up to 1000 ml. Final |
| | concentration TWEEN ®-80: 50 mM and |
| | 0.005% |
| Picrylsulfonic acid (TNBS) | Sigma P-2297 (5% solution in water) |
| Reagent A | 45.4 g Na₂B₄O₇•10 H2O (Merck 6308) and |
| | 15 ml of 4N NaOH dissolved together to a |
| | final volume of 1000 ml (by heating if |
| | needed) |
| Reagent B | 35.2 g NaH₂PO₄•1H₂O (Merck 6346) and |
| | 0.6 g Na₂SO₃ (Merck 6657) dissolved |
| | together to a final volume of 1000 ml. |

### Method

To prepare the substrate, 4 g DMC was dissolved in 400 ml PIPES buffer. The filtered culture supernatants were diluted with PIPES buffer. Then, 10 μl of each diluted supernatant were added to 200 μl substrate in the wells of a MTP. The MTP was covered with tape, shaken for a few seconds and placed in an oven at 25° C. for 30 minutes without agitation. About 15 minutes before removal of the 1^{st} plate from the oven, the TNBS reagent was prepared by mixing 1 ml TNBS solution per 50 ml of Reagent A. MTPs were filled with 60 μl TNBS Reagent A per well. The incubated plates were shaken for a few seconds, after which 10 μl was transferred to the MTPs with TNBS Reagent A. The plates were covered with tape and shaken for 20 minutes in a bench shaker (BMG Thermostar) at room temperature and 500 rpm. Finally, 200 μl Reagent B was added to the wells, mixed for 1 minute on a shaker, and the absorbance at 405 nm was determined using a MTP reader.

The obtained absorbance value was corrected for the blank value (i.e., substrate without enzyme). The resulting absorbance was a measure of the hydrolytic activity. The (arbitrary) specific activity of a sample was calculated by dividing the absorbance and the determined protein concentration.

4. 2-Aminobenzoyl-L-alanylglycyl-L-leucyl-L-alamino-4-nitrobenzylamide Assay (Abz-AGLA-Nba)

The method provided below provides a degree of technical detail that yields reproducible protease assay data independent of time and place. While the assay can be adapted to a given laboratory condition, any data obtained through a modified procedure must be reconciled with results produced by the original method. Neutral metalloproteases cleave the peptide bond between glycine and leucine of 2-aminobenzoyl-L-alanylglycyl-L-leucyl-L-alamino-4-nitrobenzylamide (Abz-AGLA-Nba). Free 2-aminobenzoyl-L-alanylglycine (Abz-AG) in solution has a fluorescence emission maximum at 415 nm with an excitation maximum of 340 nm. Fluorescence of Abz-AG is quenched by nitrobenzylamide in the intact Abz-AGLA-Nba molecule.

In these experiments, the liberation of Abz-AG by protease cleavage of Abz-AGLA-Nba was monitored by fluorescence spectroscopy (Ex. 340/Em. 415). The rate of appearance of Abz-AG was a measure of proteolytic activity. Assays were performed under non-substrate limited initial rate conditions.

A microplate mixer with temperature control (e.g., Eppendorf Thermomixer) was required for reproducible assay results. The assay solutions were incubated to desired temperature (e.g., 25° C.) in the microplate mixer prior to enzyme addition. Enzyme solutions were added to the plate in the mixer, mixed vigorously and rapidly transferred to the plate reader.

A spectrofluorometer with capability of continuous data recording, linear regression analysis, and temperature control was required (e.g., SpectraMax M5, Gemini EM, Molecular Devices). The reader was always maintained at the desired temperature (e.g., 25° C.). The reader was set for top-read fluorescence detection and the excitation was set to 350 nm and emission to 415 nm without the use of a cut-off filter. The PMT was set to medium sensitivity and 5 readings per well. Autocalibration was turned on, but only to calibrate before the first reading. The assay was measured for 3 minutes with the reading interval minimized according to the number of wells selected to be monitored. The reader was set to calculate the rate of milli-RFU/min (thousandths of relative fluorescence units per minute). The number of readings used to calculate the rate (Vmax points) was set to the number equivalent to 2 minutes, as determined by the reading interval (e.g., a reading every 10 seconds would use 12 points to calculate the rate). The max RFU was set to 50,000.

All pipeting of enzyme and substrate stock solutions were done with positive displacement pipets (Rainin Microman). Buffer, assay, and enzyme working solutions were pipetted by single or multi-channel air-displacement pipets (Rainin LTS) from tubes, reagent reservoirs or stock microplates. A repeater pipet (Eppendorf) finds use in transferring the assay solution to microplate wells when few wells are used, to minimize reagent loss. Automated pipetting instruments such as the Beckman FX or Cybio Cybi-well also find use in transferring enzyme solutions from a working stock microplate to the assay microplate in order to initiate an entire microplate at once.

### Reagents and Solutions:

### 52.6 mM MES/NaOH, 2.6 mM CaCl₂, pH 6.5—MES Buffer

MES acid (10.28 g) and 292 mg anhydrous CaCl₂ were dissolved in approximately 900 mL purified water. The solution was titrated with NaOH to pH 6.5 (at 25° C. or with temperature adjustment pH probe). The pH-adjusted buffer was made up to 1 L total volume. The final solution was filtered through a 0.22 μm sterile filter and kept at room temperature.

### 48 mM Abz-AGLA-Nba in DMF-Abz-AGLA-Nba Stock

Approximately 28 mg of Abz-AGLA-Nba was placed in a small tube. It was dissolved in DMF (volume will vary depending upon Abz-AGLA-Nba massed) and vortexed for several minutes. The solution was stored at room temperature shielded from light.

### 50 mM MES, 2.5 mM CaCl₂, 5% DMF, 2.4 mM Abz-AGLA-Nba pH 6.5—Assay Solution

One mL Abz-AGLA-Nba stock was added to 19 mL MES Buffer and vortexed. The solution was stored at room temperature shielded from light.

### 50 mM MES, 2.5 mM CaCl₂, pH 6.5—Enzyme Dilution Buffer

This buffer was produced by adding 5 mL purified water to 95 mL MES Buffer.

### 50 mM MES, 2.5 mM CaCl₂, 5% DMF, pH 6.5—Substrate Dilution Buffer

Five mL pure DMF were added to 95 mL MES Buffer. This buffer was used to determine kinetic parameters.

### Enzyme Solutions

The enzyme stock solutions were diluted with enzyme dilution buffer to a concentration of approximately 1 ppm (1 ug/mL). MULTIFECT® neutral protease (wild-type NprE) was diluted to concentrations below 6 ppm (6 ug/mL). Serial dilutions were preferred. Solutions were stable at room temperature for 1 hour, but for longer term storage, the solutions were maintained on ice.

### Procedure

First all buffers, stock, and working solutions were prepared. Each enzyme dilution was assayed in triplicate, unless otherwise indicated. When not completely full, the enzyme working solution stock microplate was arranged in full vertical columns starting from the left of the plate (to accommodate the plate reader). The corresponding assay plate was similarly set up. The microplate spectrofluorometer was set up as previously described.

First, a 2000 □L aliquot of assay solution were placed in the wells of a 96-well microplate. The plate was incubated for 10 min at 25° C. in a temperature controlled microplate mixer, shielded from light. The assay was initiated by transferring 10 uL of the working enzyme solutions from the stock microplate to the assay microplate in the mixer. Optimally, 96-well pipetting head finds use, or an 8-well multi-channel pipet was used to transfer from the left-most column first. The solutions were vigorously mixed for 15 seconds (900 rpm in Eppendorf Thermomixer) Immediately, the assay microplate was transferred to the microplate spectrofluorometer and recording of fluorescence measurements at excitation of 350 nm and emission of 415 nm were begun. The spectrofluorometer software calculated the reaction rates of the increase in fluorescence for each well to a linearly regressed line of milli-RFU/min In some experiments, a second plate was placed in the microplate mixer for temperature equilibration while the first plate was being read.

The rate initial velocities were linear with respect to product concentration (i.e., liberated 2-aminobenzoyl fluorescence) up to 0.3 mM product, which corresponded to approximately 50,000 RFU in a solution starting at 2.3 mM Abz-AGLA-Nba with background fluorescence of approximately 22,000 RFU. Abz-AGLA-Nba was dissolved in DMF and was been used the day it was prepared.

5. suc-AAPF-pNA Assay

Serine protease activity was determined by measuring cleavage of a N-succinyl-L-Ala-L-L-Ala-L-Pro-L-Phe-p-nitroanilide (suc-AAPF-pNA) substrate. The assay is based upon the cleavage by proteases of the amide bond between phenylalanine and p-nitroaniline of the N-succinyl reagent. P-nitroaniline is monitored spectrophotometrically at 410 nm and the rate of the appearance of p-nitroaniline is a measure of proteolytic activity. A protease unit is defined as the amount of protease enzyme that increases absorbance at 410 nm by 1 absorbance unit (AU)/min of a standard solution of 1.6 mM suc-AAPF-pNA in 0.1 M Tris Buffer at 25° C. in a cuvette with a 1 cm path length.

### C. Detergent Compositions:

In the exemplified detergent compositions, the enzymes levels are expressed by pure enzyme by weight of the total composition and unless otherwise specified, the detergent ingredients are expressed by weight of the total compositions. The abbreviated component identifications therein have the following meanings:

### Abbreviation Ingredient

LAS: Sodium linear C₁₁₋₁₃ alkyl benzene sulfonate.
NaC16-17HSAS: Sodium C₁₆₋₁₇ highly soluble alkyl sulfate
TAS: Sodium tallow alkyl sulphate.
CxyAS: Sodium C₁ₓ-C_{1y} alkyl sulfate.
CxyEz: C₁ₓ-C_{1y} predominantly linear primary alcohol condensed with an average of z moles of ethylene oxide.
CxyAEzS: C₁ₓ-C_{1y} sodium alkyl sulfate condensed with an average of z moles of ethylene oxide. Added molecule name in the examples.
Nonionic: Mixed ethoxylated/propoxylated fatty alcohol e.g. Plurafac LF404 being an alcohol with an average degree of ethoxylation of 3.8 and an average degree of propoxylation of 4.5.
QAS: R₂.N+(CH₃)₂(C₂H₄OH) with R₂═C₁₂-C₁₄.
Silicate: Amorphous Sodium Silicate (SiO₂:Na₂O ratio=1.6-3.2:1).
Metasilicate: Sodium metasilicate (SiO₂:Na₂O ratio=1.0).
Zeolite A: Hydrated Aluminosilicate of formula Na₁₂(AlO₂SiO₂)₁₂. 27H₂O
SKS-6: Crystalline layered silicate of formula δ-Na₂Si₂O₅.
Sulfate: Anhydrous sodium sulphate.
STPP: Sodium Tripolyphosphate.
MA/AA: Random copolymer of 4:1 acrylate/maleate, average molecular weight about 70,000-80,000.
AA: Sodium polyacrylate polymer of average molecular weight 4,500.
Polycarboxylate: Copolymer comprising mixture of carboxylated monomers such as acrylate, maleate and methyacrylate with a MW ranging between 2,000-80,000 such as Sokolan commercially available from BASF, being a copolymer of acrylic acid, MW4,500.
BB1: 3-(3,4-Dihydroisoquinolinium)propane sulfonate
BB2: 1-(3,4-dihydroisoquinolinium)-decane-2-sulfate
PB 1: Sodium perborate monohydrate.
PB4: Sodium perborate tetrahydrate of nominal formula NaBO₃.4H₂O.
Percarbonate: Sodium percarbonate of nominal formula 2Na₂CO₃.3H₂O₂.
TAED: Tetraacetyl ethylene diamine.
NOBS: Nonanoyloxybenzene sulfonate in the form of the sodium salt.
DTPA: Diethylene triamine pentaacetic acid.
HEDP: 1,1-hydroxyethane diphosphonic acid.
DETPMP: Diethyltriamine penta (methylene) phosphonate, marketed by Monsanto under the Trade name Dequest 2060.
EDDS: Ethylenediamine-N,N′-disuccinic acid, (S,S) isomer in the form of its sodium salt
Diamine: Dimethyl aminopropyl amine; 1,6-hezane diamine; 1,3-propane diamine; 2-methyl-1,5-pentane diamine; 1,3-pentanediamine; 1-methyl-diaminopropane.
DETBCHD 5,12-diethyl-1,5,8,12-tetraazabicyclo [6,6,2]hexadecane, dichloride, Mn(II) SALT
PAAC: Pentaamine acetate cobalt(III) salt.
Paraffin: Paraffin oil sold under the tradename Winog 70 by Wintershall.
Paraffin Sulfonate: A Paraffin oil or wax in which some of the hydrogen atoms have been replaced by sulfonate groups.
Aldose oxidase: Oxidase enzyme sold under the tradename Aldose Oxidase by Novozymes A/S
Galactose oxidase: Galactose oxidase from Sigma
nprE: The recombinant form of neutral metalloprotease expressed in Bacillus subtilis.
PMN: Purified neutral metalloprotease from Bacillus amyloliquefacients.
Amylase Amylolytic enzyme sold under the tradename PURAFECT® Ox described in WO 94/18314, WO96/05295 sold by Genencor; NATALASE®, TERMAMYL®, FUNGAMYl® and DURAMYL®, all available from Novozymes A/S.
Lipase: Lipolytic enzyme sold under the tradename LIPOLASE®, LIPOLASE® Ultra by Novozymes A/S and Lipomax™ by Gist-Brocades.
Cellulase: Cellulytic enzyme sold under the tradename Carezyme, Celluzyme and/or Endolase by Novozymes A/S.
Pectin Lyase: PECTAWAY® and PECTAWASH® available from Novozymes A/S.
PVP: Polyvinylpyrrolidone with an average molecular weight of 60,000
PVNO: Polyvinylpyridine-N-Oxide, with an average molecular weight of 50,000.
PVPVI: Copolymer of vinylimidazole and vinylpyrrolidone, with an average molecular weight of 20,000.
Brightener 1: Disodium 4,4′-bis(2-sulphostyryl)biphenyl.
Silicone antifoam: Polydimethylsiloxane foam controller with siloxane-oxyalkylene copolymer as dispersing agent with a ratio of said foam controller to said dispersing agent of 10:1 to 100:1.
Suds Suppressor: 12% Silicone/silica, 18% stearyl alcohol, 70% starch in granular form.
SRP 1: Anionically end capped poly esters.
PEG X: Polyethylene glycol, of a molecular weight of x.
PVP K60®: Vinylpyrrolidone homopolymer (average MW 160,000)
Jeffamine® ED-2001: Capped polyethylene glycol from Huntsman
Isachem® AS: A branched alcohol alkyl sulphate from Enichem
MME PEG (2000): Monomethyl ether polyethylene glycol (MW 2000) from Fluka Chemie AG.
DC3225C: Silicone suds suppresser, mixture of Silicone oil and Silica from Dow Corning.
TEPAE: Tetreaethylenepentaamine ethoxylate.
BTA: Benzotriazole.
Betaine: (CH₃)₃N⁺CH₂COO⁻
Sugar: Industry grade D-glucose or food grade sugar
CFAA: C₁₂-C₁₄ alkyl N-methyl glucamide
TPKFA: C₁₂-C₁₄ topped whole cut fatty acids.
Clay: A hydrated aluminum silicate in a general formula Al₂O₃SiO₂.xH₂O. Types: Kaolinite, montmorillonite, atapulgite, illite, bentonite, halloysite.
pH: Measured as a 1% solution in distilled water at 20° C.

### Example 2

### NprE Protease Production in B. subtilis

In this Example, experiments conducted to produce NprE protease in B. subtilis are described. In particular, the methods used in the transformation of plasmid pUBnprE into B. subtilis are provided. Transformation was performed as known in the art (See e.g., WO 2002/014490, and WO 2007/044993 both incorporated herein by reference). The DNA sequence (nprE leader, nprE pro and nprE mature DNA sequence from B. amyloliquefaciens) provided below, encodes the NprE precursor protein:

(SEQ ID NO: 1)
GTGGGTTTAGGTAAGAAATTGTCTGTTGCTGTCGCCGCTTCCTTTATGAG
TTTAACCATCAGTCTGCCGGGTGTTCAGGCCGCTGAGAATCCTCAGCTTA
AAGAAAACCTGACGAATTTTGTACCGAAGCATTCTTTGGTGCAATCAGAA
TTGCCTTCTGTCAGTGACAAAGCTATCAAGCAATACTTGAAACAAAACGG
CAAAGTCTTTAAAGGCAATCCTTCTGAAAGATTGAAGCTGATTGACCAAA
CGACCGATGATCTCGGCTACAAGCACTTCCGTTATGTGCCTGTCGTAAAC
GGTGTGCCTGTGAAAGACTCTCAAGTCATTATTCACGTCGATAAATCCAA
CAACGTCTATGCGATTAACGGTGAATTAAACAACGATGTTTCCGCCAAAA
CGGCAAACAGCAAAAAATTATCTGCAAATCAGGCGCTGGATCATGCTTAT
AAAGCGATCGGCAAATCACCTGAAGCCGTTTCTAACGGAACCGTTGCAAA
CAAAAACAAAGCCGAGCTGAAAGCAGCAGCCACAAAAGACGGCAAATACC
GCCTCGCCTATGATGTAACCATCCGCTACATCGAACCGGAACCTGCAAAC
TGGGAAGTAACCGTTGATGCGGAAACAGGAAAAATCCTGAAAAAGCAAAA
CAAAGTGGAGCATGCCGCCACAACCGGAACAGGTACGACTCTTAAAGGAA
AAACGGTCTCATTAAATATTTCTTCTGAAAGCGGCAAATATGTGCTGCGC
GATCTTTCTAAACCTACCGGAACACAAATTATTACGTACGATCTGCAAAA
CCGCGAGTATAACCTGCCGGGCACACTCGTATCCAGCACCACAAACCAGT
TTACAACTTCTTCTCAGCGCGCTGCCGTTGATGCGCATTACAACCTCGGC
AAAGTGTATGATTATTTCTATCAGAAGTTTAATCGCAACAGCTACGACAA
TAAAGGCGGCAAGATCGTATCCTCCGTTCATTACGGCAGCAGATACAATA
ACGCAGCCTGGATCGGCGACCAAATGATTTACGGTGACGGCGACGGTTCA
TTCTTCTCACCTCTTTCCGGTTCAATGGACGTAACCGCTCATGAAATGAC
ACATGGCGTTACACAGGAAACAGCCAACCTGAACTACGAAAATCAGCCGG
GCGCTTTAAACGAATCCTTCTCTGATGTATTCGGGTACTTCAACGATACT
GAGGACTGGGATATCGGTGAAGATATTACGGTCAGCCAGCCGGCTCTCCG
CAGCTTATCCAATCCGACAAAATACGGACAGCCTGATAATTTCAAAAATT
ACAAAAACCTTCCGAACACTGATGCCGGCGACTACGGCGGCGTGCATACA
AACAGCGGAATCCCGAACAAAGCCGCTTACAATACGATTACAAAAATCGG
CGTGAACAAAGCGGAGCAGATTTACTATCGTGCTCTGACGGTATACCTCA
CTCCGTCATCAACTTTTAAAGATGCAAAAGCCGCTTTGATTCAATCTGCG
CGGGACCTTTACGGCTCTCAAGATGCTGCAAGCGTAGAAGCTGCCTGGAA
TGCAGTCGGATTGTAA

In the above sequence, bold indicates the DNA that encodes the mature NprE protease, standard font indicates the leader sequence (nprE leader), and underlined indicates the pro sequences (nprE pro). The amino acid sequence (NprE leader, NprE pro and NprE mature DNA sequence) provided below (SEQ ID NO:2), corresponds to the full length NprE precursor protein. In this sequence, underlined indicates the pro sequence and bold indicates the mature NprE protease.

(SEQ ID NO: 2)
MGLGKKLSVAVAASFMSLTISLPGVQAAENPQLKENLTNFVPKHSLVQSE
LPSVSDKAIKQYLKQNGKVFKGNPSERLKLIDQTTDDLGYKHFRYVPVVN
GVPVKDSQVIIHVDKSNNVYAINGELNNDVSAKTANSKKLSANQALDHAY
KAIGKSPEAVSNGTVANKNKAELKAAATKDGKYRLAYDVTIRYIEPEPAN
WEVTVDAETGKILKKQNKVEHAATTGTGTTLKGKTVSLNISSESGKYVLR
DLSKPTGTQIITYDLQNREYNLPGTLVSSTTNQFTTSSQRAAVDAHYNLG
KVYDYFYQKFNRNSYDNKGGKIVSSVHYGSRYNNAAWIGDQMIYGDGDGS
FFSPLSGSMDVTAHEMTHGVTQETANLNYENQPGALNESFSDVFGYFNDT
EDWDIGEDITVSQPALRSLSNPTKYGQPDNFKNYKNLPNTDAGDYGGVHT
NSGIPNKAAYNTITKIGVNKAEQIYYRALTVYLTPSSTFKDAKAALIQSA
RDLYGSQDAASVEAAWNAVGL

The mature NprE sequence is set forth as SEQ ID NO:3. This sequence was used as the basis for making the variant libraries described herein.

(SEQ ID NO: 3)
AATTGTGTTLKGKTVSLNISSESGKYVLRDLSKPTGTQIITYDLQNREYN
LPGTLVSSTTNQFTTSSQRAAVDAHYNLGKVYDYFYQKFNRNSYDNKGGK
IVSSVHYGSRYNNAAWIGDQMIYGDGDGSFFSPLSGSMDVTAHEMTHGVT
QETANLNYENQPGALNESFSDVFGYFNDTEDWDIGEDITVSQPALRSLSN
PTKYGQPDNFKNYKNLPNTDAGDYGGVHTNSGIPNKAAYNTITKIGVNKA
EQIYYRALTVYLTPSSTFKDAKAALIQSARDLYGSQDAASVEAAWNAVGL

The pUBnprE expression vector was constructed by amplifying the nprE gene from the chromosomal DNA of B. amyloliquefaciens by PCR using two specific primers:

Oligo AB1740:
(SEQ ID NO: 4)
CTGCAGGAATTCAGATCTTAACATTTTTCCCCTATCATTTTTCCCG
Oligo AB1741:
(SEQ ID NO: 5)
GGATCCAAGCTTCCCGGGAAAAGACATATATGATCATGGTGAAGCC

PCR was performed on a thermocycler with Phusion High Fidelity DNA polymerase (Finnzymes. The PCR mixture contained 10 μl 5× buffer (Finnzymes Phusion), 1 μl 10 mM dNTP's, 1.5 μl DMSO, 1 μl of each primer, 1 μl Finnzymes Phusion DNA polymerase, 1 μl chromosomal DNA solution 50 ng/μl, 34.5 μl MilliQ water. The following protocol was used:

PCR protocol:

1) 30 sec 98° C.;

2) 10 sec 98° C.;

3) 20 sec 55° C.;

4) 1 min 72° C.;

5) 25 cycles of steps 2 to 4; and

6) 5 min 72° C.

This resulted in a 1.9 kb DNA fragment, which was digested using BglII and BclI DNA restriction enzymes. The multicopy Bacillus vector pUB110 (See e.g., Gryczan, J Bacteriol, 134:318-329, 1978) was digested with BamHI. The PCR fragment×BglII×BclI was then ligated in the pUB110×BamHI vector to form pUBnprE expression vector.

pUBnprE was transformed into a B. subtilis (ΔaprE, ΔnprE, oppA, ΔspoIIE, degUHy32, ΔamyE::(xylR,pxylA-comK) strain. Transformation into B. subtilis was performed as described in WO 02/14490, incorporated herein by reference. Selective growth of B. subtilis transformants harboring the pUBnprE vector was performed in shake flasks containing 25 ml MBD medium (a MOPS based defined medium), with 20 mg/L neomycin. MBD medium was made essentially as known in the art (See, Neidhardt et al., J Bacteriol, 119: 736-747, 1974), except that NH₄Cl₂, FeSO₄, and CaCl₂ were left out of the base medium, 3 mM K₂HPO₄ was used, and the base medium was supplemented with 60 mM urea, 75 g/L glucose, and 1% soytone. Also, the micronutrients were made up as a 100× stock containing in one liter, 400 mg FeSO₄.7H₂O, 100 mg MnSO₄.H₂O, 100 mg ZnSO₄.7H₂O, 50 mg CuCl₂.2H₂O, 100 mg CoCl₂.6H₂O, 100 mg NaMoO₄.2H₂O, 100 mg Na₂B₄O₇.10H₂O, 10 ml of 1M CaCl₂, and 10 ml of 0.5 M sodium citrate. The culture was incubated for three days at 37° C. in an incubator/shaker (Infors). This culture resulted in the production of secreted NprE protease with proteolytic activity as demonstrated by protease assays. Gel analysis was performed using NuPage Novex 10% Bis-Tris gels (Invitrogen, Catalog No. NP0301BOX). To prepare samples for analysis, 2 volumes of supernatant were mixed with 1 volume 1M HCl, 1 volume 4×LDS sample buffer (Invitrogen, Catalog No. NP0007), and 1% PMSF (20 mg/ml). The samples were subsequently heated for 10 minutes at 70° C. Then, 25 μL of each sample were loaded onto the gel, together with 10 μL of SeeBlue plus 2 pre-stained protein standards (Invitrogen, Catalog No. LC5925). The results clearly demonstrated that the nprE cloning strategy described in this example is suitable for production of active NprE in B. subtilis.

### Example 3

### Generation of Site Evaluation Libraries (SELs)

In this Example, methods used in the construction of nprE SELs are described.

Generation of nprE SELs—Method I

The pUBnprE vector, containing the nprE expression cassette described above, served as template DNA. This vector contains a unique BglII restriction site, which was utilized in the site evaluation library construction. Briefly, to construct a nprE site evaluation library, three PCR reactions were performed, including two mutagenesis PCRs to introduce the mutated codon of interest in the mature nprE DNA sequence and a third PCR used to fuse the two mutagenesis PCRs in order to construct the pUBnprE expression vector including the desired mutated codon in the mature nprE sequence.

The method of mutagenesis was based on the codon-specific mutation approach, in which the creation of all possible mutations at a time in a specific DNA triplet was performed using a forward and reverse oligonucleotide primer with a length of 25 to 45 nucleotides enclosing a specific designed triple DNA sequence NNS (N=A, C, T or G; and S═C or G) that corresponded with the sequence of the codon to be mutated and guaranteed random incorporation of nucleotides at that specific nprE mature codon. The number listed in the primer names corresponds with the specific nprE mature codon position. Multiple sites were evaluated included. An exemplary listing of primer sequences is described in WO 2007/044993, herein incorporated by reference.

Two additional primers used to construct the site evaluation libraries contained the BglII restriction site together with a part of the pUBnprE DNA sequence flanking the BglII restriction site. The following primers were produced by Invitrogen (50 nmole scale, desalted):

| | |
|---|---|
| pUB-BglII-FW | |
| GTCAGTCAGATCTTCCTTCAGGTTATGACC; | (SEQ ID NO: 6) |
| and | |
| pUB-BglII-RV | |
| GTCTCGAAGATCTGATTGCTTAACTGCTTC. | (SEQ ID NO: 7) |

Construction of each SEL started with two primary PCR amplifications using the pUB-BglII-FW primer and a specific nprE reverse mutagenesis primer. For the second PCR, the pUB-BglII-RV primer and a specific nprE forward mutagenesis primer (equal nprE mature codon positions for the forward and reverse mutagenesis primers) were used.

The introduction of the mutations in the mature nprE sequence was performed using Phusion High-Fidelity DNA Polymerase (Finnzymes; Catalog No. F-530L). All PCRs were performed according to the Finnzymes protocol supplied with the polymerase. The PCR conditions for the primary PCRs were:

For primary PCR 1:
pUB-BglII-FW primer and a specific NPRE reverse mutagenesis primer—both 1 μL (10 μM);

For primary PCR 2:
pUB-BglII-RV primer and a specific NPRE forward mutagenesis primer—both 1 μL (10 μM); together with

| | | |
|---|---|---|
| 5 × Phusion HF buffer | 10 | μL |
| 10 mM dNTP mixture | 1 | μL |
| Phusion DNA polymerase | 0.75 | μL (2 units/μL) |
| DMSO, 100% | 1 | μL |
| pUBnprE template DNA | 1 | μL (0.1-1 ng/μL) |
| Distilled, autoclaved water | up to 50 | μL |

The PCR program was: 30 seconds 98° C., 30× (10 seconds 98° C., 20 seconds 55° C., 1.5 minute 72° C.) and 5 min 72° C., performed in a PTC-200 Peltier thermal cycle (MJ Research). The PCR experiments resulted in two fragments of approximately 2 to 3 kB, which had about 30 nucleotide base overlap around the NprE mature codon of interest. Fragments were fused in a third PCR reaction using these two aforementioned fragments and the forward and reverse BglII primers. The fusion PCR reaction was carried out in the following solution:
pUB-BglII-FW primer and pUB-BglII-RV primer—both 1 μL (10 μM) together with

| | | |
|---|---|---|
| 5 × Phusion HF buffer | 10 | μL |
| 10 mM dNTP mixture | 1 | μL |
| Phusion DNA polymerase | 0.75 | μL (2 units/μL) |
| DMSO, 100% | 1 | μL |
| primary PCR 1 reaction mix | 1 | μL |
| primary PCR 2 reaction mix | 1 | μL |
| Distilled, autoclaved water | up to 50 | μL |

The PCR fusion program was as follows: 30 seconds 98° C., 30× (10 seconds 98° C., 20 seconds 55° C., 2:40 minute 72° C.) and 5 min 72° C., in a PTC-200 Peltier thermal cycler (MJ Research).

The amplified linear 6.5 Kb fragment was purified using the Qiaquick PCR purification kit (Qiagen, Catalog No. 28106) and digested with BglII restriction enzyme to create cohesive ends on both sides of the fusion fragment:

35 μL purified linear DNA fragment

4 μL REACT® 3 buffer (Invitrogen)

1 μL BglII, 10 units/ml (Invitrogen)

Reaction conditions: 1 hour, 30° C.

Ligation of the BglII digested and purified using Qiaquick PCR purification kit (Qiagen, Catalog No. 28106) fragment results in circular and multimeric DNA containing the desired mutation:

30 μL of purified BglII digested DNA fragment

8 μL T4 DNA Ligase buffer (Invitrogen Catalog No. 46300-018)

1 μL T4 DNA Ligase, 1 unit/μL (Invitrogen Catalog No. 15224-017)

Reaction conditions: 16-20 hours, 16° C.

Subsequently, the ligation mixture was transformed into a B. subtilis (ΔaprE, ΔnprE, oppA, ΔspoIIE, degUHy32, ΔamyE::(xylR,pxylA-comb) strain. Transformation to B. subtilis was performed as described in WO 02/14490, incorporated herein by reference. For each library, 96 single colonies were picked and grown in MOPS media with neomycin and 1.25 g/L yeast extract for sequence analysis (BaseClear) and screening purposes. Each library included a maximum of 19 nprE site-specific variants.

The variants were produced by growing the B. subtilis SEL transformants in 96 well MTP at 37° C. for 68 hours in MBD medium with 20 mg/L neomycin and 1.25 g/L yeast extract.

Generation of nprE SELs—Method II

Alternative methods to generate nprE SELs are also described. These methods are suitable for production of SELs of other enzymes of interest. As above, the pUBnprE vector containing the nprE expression cassette, served as the template DNA source for the generation of nprE SELs and NprE variants. The major difference between the two methods is that this method requires amplification of the entire vector using complementary site-directed mutagenic primers.

### Materials:

Bacillus strain containing the pUBnprE vector
Qiagen Plasmid Midi Kit (Qiagen Catalog No. 12143)
Ready-Lyse Lysozyme (Epicentre Catalog No. R1802M)
dam Methylase Kit (New England Biolabs Catalog No. MO222L)
Zymoclean Gel DNA Recovery Kit (Zymo Research Catalog No. D4001)
nprE site-directed mutagenic primers, 100 nmole scale, 5′ Phosphorylated, PAGE purified (Integrated DNA Technologies, Inc.)
QUIKCHANGE® Multi Site-Directed Mutagenesis Kit (Stratagene Catalog No. 200514)
MJ Research PTC-200 Peltier Thermal Cycler (Bio-Rad Laboratories)
1.2% agarose E-gels (Invitrogen Catalog No. G5018-01)
TempliPhi Amplification Kit (GE Healthcare Catalog No. 25-6400-10)
Competent B. subtilis cells (ΔaprE, ΔnprE, oppA, ΔspoIIE, degUHy32, ΔamyE::(xylR,pxylA-comK)

### Methods:

To obtain the pUBnprE plasmids containing one mutation (identified through nprE SEL screening as described above in Example 3 and in WO 2007/044993, herein incorporated by reference), a single colony of each Bacillus strain of interest was used to inoculate a 5 ml LB+10 ppm neomycin tube (e.g., starter culture). The culture was grown at 37° C., with shaking at 225 rpm for 6 hours. Then 100 ml of fresh LB+10 ppm neomycin were inoculated with 1 ml of the starter culture. This culture was grown overnight at 37° C., with shaking at 225 rpm. Following this incubation, the cell pellet was harvested by sufficient centrifugation to provide a cell pellet. The cell pellet was resuspended in 10 ml Buffer P1 (Qiagen Plasmid Midi Kit). Then, 10 □l of Ready-Lyse Lysozyme was added to the resuspended cell pellet and incubated at 37° C. for 30 min The Qiagen Plasmid Midi Kit protocol was continued using 10 ml of Buffer P2 and P3 to account for the increased volume of cell culture. After isolation from Bacillus of each pUBnprE plasmid containing a single nprE mutation, the concentration of each plasmid was determined. The plasmids were then dam methylated using the dam Methylase Kit (New England Biolabs) per the manufacturer's instructions, to methylate approximately 2 μg of each pUBnprE plasmid per tube. The Zymoclean Gel DNA recovery kit was used to purify and concentrate the dam-methylated pUBnprE plasmids. The dam-methylated pUBnprE plasmids were then quantitated and diluted to a working concentration of 50 ng/μl for each. Mixed site-directed mutagenic primers were prepared separately for each reaction. For example, using pUBnprE T14R plasmid as the template source, the mixed site-directed mutagenic primer tube would contain 10 μl of nprE-S23R, 10 μl nprE-G24R, 10 μl nprE-N46K, and 10 μl nprE-T54R (all primers at 10 μM each). A PCR reaction using the QuikChange Multi Site-Directed Mutagenesis Kit (Stratagene) was performed following the manufacturer's instructions (e.g., 1 μl dam methylated pUBnprE plasmid containing one mutation (50 ng/μl), 2 μl nprE site-directed mutagenic primers (10 μM), 2.5 μl 10× QuikChange Multi Reaction buffer, 1 μl dNTP Mix, 1 μl QuikChange Multi enzyme blend (2.5 U/μl), and 17.5 μl distilled, autoclaved water, to provide a 25 μl total reaction mix. The nprE variant libraries were amplified using the following conditions: 95° C., for 1 min (1^{st} cycle only), followed by 95° C. for 1 min, 55° C. for 1 min, 65° C. for 13.5 min, and repeat cycling 29 times. The reaction product was stored at 4° C. overnight. Then, the reaction mixture underwent DpnI digest treatment (supplied with QUIKCHANGE® Multi Site-Directed Mutagenesis Kit) to digest parental pUB-nprE plasmid, using the manufacturer's protocol (i.e., 1.5 μl DpnI restriction enzyme was added to each tube and incubated at 37° C. for 3 hours; 2 μl of DpnI-digested PCR reaction was then analyzed on a 1.2% E-gel to ensure PCR reaction worked and that parental template was degraded. TempliPhi rolling circle amplification was then used to generate large amounts of DNA for increasing library size of the nprE multi variants, using the manufacturer's protocol (i.e., 1 μl DpnI treated QuikChange Multi Site-Directed Mutagenesis PCR, 5 μl TempliPhi Sample Buffer, 5 μl TempliPhi Reaction Buffer, and 0.2 μl TempliPhi Enzyme Mix, for an ˜11 μl total reaction; incubated at 30° C. for 3 hours; the TempliPhi reaction was diluted by adding 200 μl distilled, autoclaved water and briefly vortexed. Then, 1.5 μl of diluted TempliPhi material was transformed into competent B. subtilis cells, and nprE multi variants were selected for using LA+10 ppm Neomycin +1.6% skim milk plates. Colonies were picked and then sequenced to identify the different nprE variant library combinations.

Integrated DNA Technologies synthesized all of the primers (100 nmole scale, 5′-phosphorylated, and PAGE purified) used for mutagenesis. Sites evaluated included: 4, 12, 13, 23, 45, 49, 50, 54, 59, 60, 65, 82, 90, 110, 119, 128, 129, 130, 135, 136, 137, 138, 139, 140, 151, 152, 155, 179, 190, 197, 198, 199, 204, 205, 214, 216, 217, 218, 219, 220, 221, 222, 224, 243, 244, 260, 261, 263, 265, 269, 273, 282, 285, 286, 289, 293, 296, 297 and 299. Exemplary mutagenesis primers are described in WO 2007/044993, herein incorporated by reference.

### Example 4

### Expression, Fermentation, and Purification of Variant Proteases

This Example describes the methods used to express, ferment and purify the proteases of the transformed B. subtilis.

### Neutral Metalloproteases

The recombinant Bacillus subtilis was cultivated by conventional batch fermentation in a nutrient medium. One glycerol vial of a B. subtilis culture (containing the B. amyloliquefaciens neutral metalloprotease or a variant thereof) was used to inoculate 600 ml of SBG 1% medium containing 200 mg/L chloramphenicol. The cultures were grown for 36-48 hours at 37° C. Alternative methods employed involved the growth of recombinant B. subtilis in defined media for 60 hours at 35° C. The culture fluid was subsequently recovered by centrifugation at 12,000 rpm, as known in the art (SORVALL® centrifuge model RC5B). The secreted neutral metalloproteases were isolated from the culture fluid and concentrated approximately 10-fold using an Amicon filter system 8400 with a BES (polyethersulfone) 10 kDa cutoff.

The concentrated supernatant was dialyzed overnight at 4° C. against 25 mM MES buffer, pH 5.4, containing 1 mM CaCl₂. The dialyzate was then loaded onto a cation-exchange column Poros HS20 (Applied Biosystems column with a total volume ˜83 mL; binding capacity ˜4.5 g protein/mL column; waters). The column was pre-equilibrated with 25 mM MES buffer, pH 5.4, containing 1 mM CaCl₂. The bound protein was eluted using a pH and salt gradient from 25 mM MES, pH 5.4, 1 mM CaCl₂ to 50 mM MES, pH 6.2, 2 mM CaCl₂ and 100 mM NaCl. Elution of the protein was between pH 5.8 and 6.0. The pure protein was concentrated and buffer-exchanged into 25 mM MES buffer, pH 5.8, containing 2 mM CaCl₂, and 40% propylene glycol. The purity of the preparation was assessed by measuring proteolytic activity and by 10% (w/v) NU-PAGE® Novex SDS-PAGE (Invitrogen Corp.) and found to be greater than 95%.

### Example 5

### Genetic Engineering of Bacillus Host Strains

This example briefly describes the construction of multiple B. subtilis host strains used to improve production of recombinant enzymes. Exemplary methods employed the following steps:

Step 1—Deletion of Alkaline Protease and Introduction of scoC:

The alkaline protease gene (aprE) was deleted from the B. subtilis 168 derived research strain using recombinant DNA techniques (Stahl and Ferrari, J Bacteriol, 158:411-418, 1984; and Yang et al., J Bacteriol, 160:15-21, 1984). The deletion was introduced by PBS1 transduction. Although the deletion was initially created by recombinant DNA (rDNA) techniques, there was no heterologous DNA remaining after excision of the plasmid. Concurrent with the introduction of the aprE deletion, another mutation, scoC, was also introduced. This mutation has been previously described as a mutation that increases extracellular protease production (Dod et al., Molec Gene Genet, 163:45-56, 1978).

### Step 2—Deletion of Neutral Protease:

Introduction of a deletion in a second extracellular protease, the neutral protease (npr), was also accomplished using recombinant DNA techniques. A deletion was first created in the npr gene in a research strain (Yang et al., J Bacteriol, 160:15-21, 1984) before introduction of the mutated gene into the aprE [symbol] scoC strain from Step 1.

### Step 3—Removal of Threonine Auxotrophy:

Since the strain from Step 2 is auxotrophic for the amino acids histidine, threonine and tryptophan (i.e., requires these three nutrients for growth), it was transformed, using competent cell transformation, to prototrophy for threonine. Consequently, the strain derived in Step 3 no longer requires threonine for growth, but does still require tryptophan and histidine.

### Step 4—Introduction of Sporulation Mutation and Removal of Tryptophan Auxotrophy:

This step involved the introduction of a sporulation deficiency (spo⁻) mutation that does not affect enzyme production. A protease producing research strain was mutagenized with the chemical mutagen N-Nitro-N-nitrosoguanidine (NTG) as known in the art (Gerhardt (ed.) Manual of Methods for General Bacteriology, American Society for Microbiology, Washington, D.C., p. 226, 1981). Spo⁻ mutants were isolated and screened for retention of high levels of protease production, comparable to the non-mutagenized strain. To introduce the spo⁻ mutation into the strain of Step 3, chromosomal DNA was prepared from the mutagenized strain and, using competent cell transformation, both the tryptophan marker and the spo mutation were introduced into the strain from Step 3. The strain obtained in this way contains the spo⁻ mutation and no longer requires tryptophan for growth, but still requires histidine.

Step 5—Introduction of sacU(h) Gene and Removal of Histidine Auxotrophy:

The sacU(h) gene was introduced into the strain from Step 4 by PBS-1 mediated transduction. This allele of the sacU(h) gene, which is clearly different from the scoC mutation, results in the increased production of several extracellular enzymes in B. subtilis (Kunst et al., Biochemie, 56:1481-1489, 1974). The requirement for histidine was simultaneously eliminated with the introduction of the sacU(h) gene. The resulting strain is a prototrophic strain deficient in spore formation.

### Step 6—Introduction of Sporulation Control Deletion:

After the sacU(h) gene was introduced, another sporulation mutation, called the spo-3501 deletion, was introduced. This mutation was identified by transposon mutagenesis. The gene was cloned and a deletion was made and introduced into the host strain. In combination, the spo and the spo-3501 deletion sporulation mutations greatly reduce the sporulation frequency.

### Step 7—Introduction of Protease:

The B. subtilus host strain of Step 6 has been used for high level production of several enzymes. This was accomplished by transformation of the host strain with a plasmid expression vector carrying a coding region of an enzyme of interest in operable combination with a suitable promoter.

### Step 8—NTG Mutagenesis:

For enhancement of expression of the recombinant enzyme of Step 7, the transformed host was treated with NTG. A number of independent isolates were screened for the ability to produce more product than the parent strain. A highly efficient producer was isolated in this way.

### Step 9—Loopout of Gene Product:

To use the host strain created in Steps 7 or 8 as a general production host, the gene of the enzyme of interest was removed by standard techniques. This involved the loopout of the gene by homologous recombination in the Bacillus host.

### Step 10—Removal of Extracellular Protease:

The minor extra-cellular protease (Epr) was deleted from the host strain (Sloma et al., J Bacteriol, 170:5557-5563, 1988) using recombinant DNA techniques similar to those employed in the deletion of aprE. The deletion in the epr gene was confirmed by Southern hybridization.

### Step 11—Removal of Intracellular Serine Protease:

A deletion in the intracellular serine protease (ISP) gene (Koide et al., J Bacteriol, 167:110-116, 1986) was made using recombinant DNA techniques similar to those used employed in the deletion of aprE. The efficacy of the isp deletion was monitored by measuring ISP activity, as well as by Southern blot.

### Step 12—Removal of Bacillopeptidase F Protease:

A deletion in the bacillopeptidase F (BPF) gene (Sloma et al., J Bacteriol, 172:1470-1477, 1990) was made using recombinant DNA techniques similar to those used in the deletion of aprE. The deletion in the bpf gene was confirmed by Southern hybridization.

### Step 13—Removal of Amylase:

An in vitro created deletion of the wild-type amylase (amyE) gene was introduced into the B. subtilis strain of step 12. The plasmid amy/pUCTskan, which carries a disrupted amylase gene, was transformed into BG3934 by natural competence. This plasmid also bears a kanamycin-resistance gene (kanr) and a temperature sensitive origin of replication (TsOri). The kanr gene used in this work was originally isolated from the Streptococcus faecalis plasmid, pJH1 (Trieu-Coet and Courvalin, Gene, 23: 331-341, 1983).

Due to the presence of the TsOri, this plasmid integrated into the chromosome at the region of homology with the amylase gene at the non-permissive temperature (e.g., 48° C.). After integration, the strain carrying the plasmid was grown extensively at the permissive temperature in the absence of kanamycin. This allows the excision and loss of the plasmid, giving rise to either the parental strain, or to a mutant lacking the amylase gene.

### Step 14 —Removal of Wall Protease:

A deletion of the wall protease gene (wprA) was introduced into the strain of step 13 by removal of the nucleotides encoding amino-terminal WprA residues. A first plasmid was created containing the spectinomycin gene with flanking regions of the upstream region (5′) of the wprA, as well as the downstream region (3′) encoding carboxy-terminal WprA residues. A second plasmid was created containing kanamycin and a temperature sensitive origin of replication (TsOri), which permits integration at temperatures above 37° C. The second plasmid also contains the same 5′ and 3′ wprA DNA fragments as the first plasmid, although in the second plasmid the spectinomycin gene is not present to disrupt the 5′ and 3′ wprA DNA fragments.

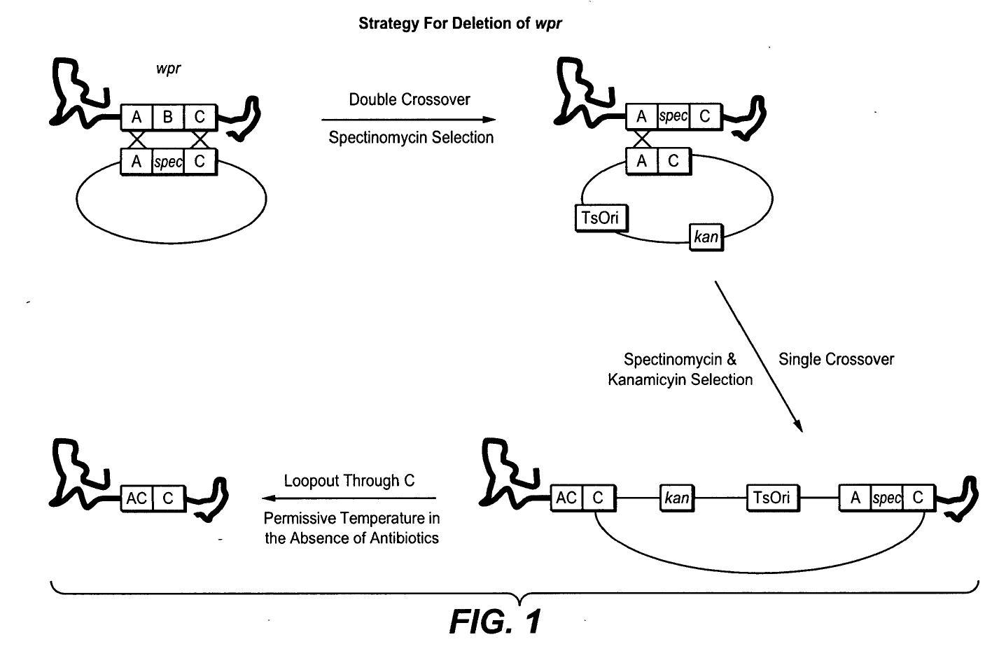

The spectinomycin-containing plasmid was first integrated into the host strain by double crossover, replacing the intact wprA gene. Next, the TsOri containing plasmid was transformed into the spectinomycin containing strain by Campbell integration. The second transformation introduces a cassette into the host chromosome containing the wprA deletion as depicted in FIG. 1. This cassette was transformed into the host using chromosomal DNA from the dual transformant, and grown under permissive temperature (30° C.) in the absence of any antibiotics. Subsequently the population was screened for clones that had lost both kanamycin and spectinomycin resistance to obtain wprA deletion mutants lacking heterologous DNA.

### Example 6

### Construction of Bacillus Host Strains Bearing Multiple Protease Gene Deletions

This example provides generic methods for the engineering of B. subtilis to eliminate production of endogenous proteases using the Cre/loxP site-specific recombination system (Palmeros et al., Gene, 247: 255-264, 2000). Exemplary methods employ the following steps:

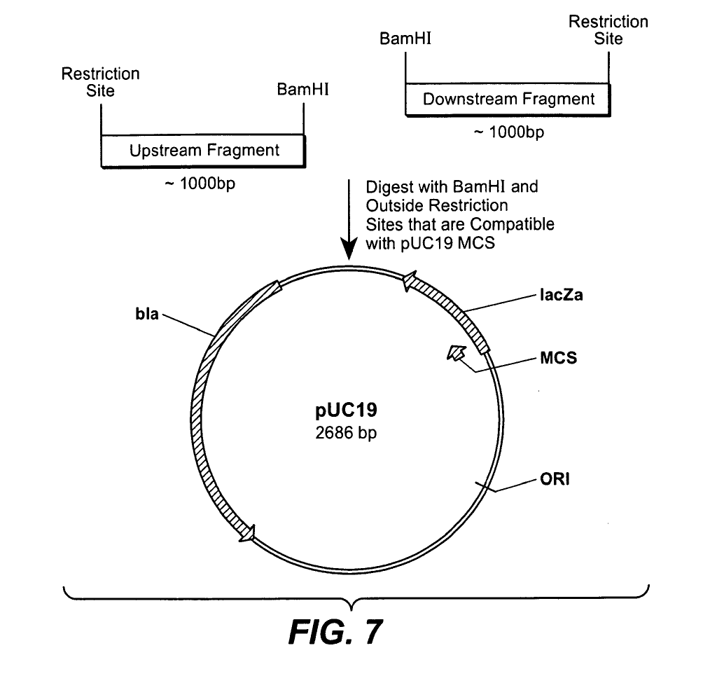

Step 1—construct a B. subtilis protease gene deletion plasmid by PCR amplifying approximately 1000 by of homologous chromosomal DNA of the upstream and downstream fragments of the proposed protease gene deletion site with convenient restriction sites engineered to the end of the primers (See e.g., FIG. 7).

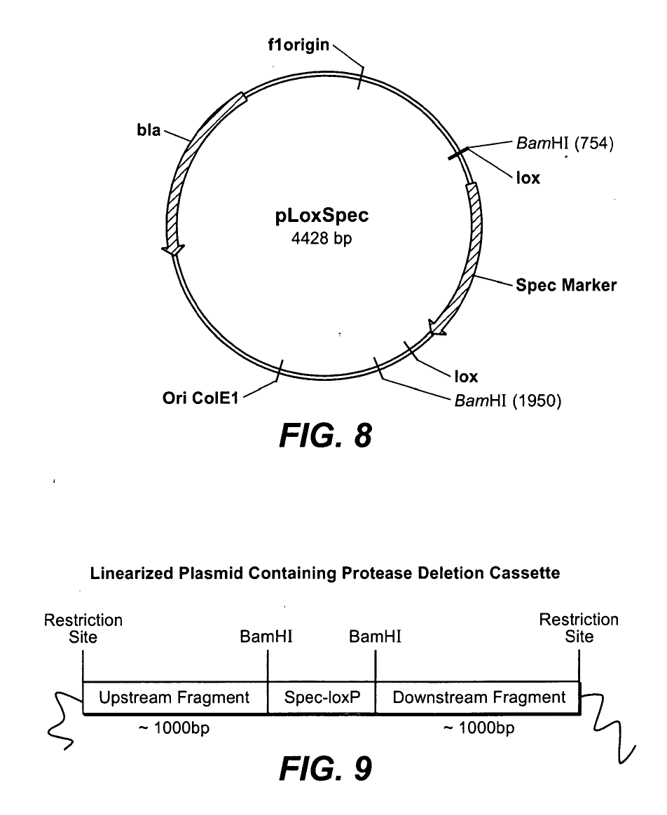

Step 2—digest the Spec-loxP fragment from pLoxSpec plasmid using BamHI (See e.g., FIG. 8).

Step 3—digest the B. subtilis protease gene deletion plasmid using BamHI, and subclone the Spec-loxP fragment into the B. subtilis protease gene deletion plasmid.

Step 4—digest the B. subtilis protease gene deletion plasmid with a restriction endonuclease that does not cut within the upstream chromosomal DNA-Spec-loxP-downstream chromosomal DNA cassette to linearize the plasmid (See e.g., FIG. 9).

Step 5—transform a B. subtilis host strain with the linearized plasmid. Any transformants obtained on selective media will have the Spec-loxP cassette integrated into the chromosome at the targeted gene deletion site via double-crossover integration.

Step 6—prepare B. subtilis competent cells of the new host strain.

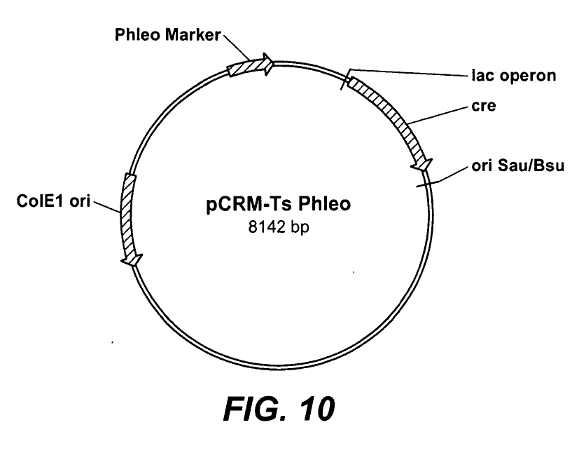

Step 7—transform the pCRM-Ts Phleo plasmid into the new host strain (See e.g., FIG. 10) and select on phleomycin plates at 30° C.

Step 8—inoculate an LB shake flask with a single phleomycin-resistant colony in the absence of antibiotic (e.g., non-selective pressure) and grow at 42° C. for 6 hours.

Step 9—plate the culture on LB agar plates in the absence of antibiotics and incubate at 37° C.

Step 10—pick and patch colonies onto fresh plates (one with and the other without antibiotic) to identify colonies having the proposed gene deletion, but lacking the heterologous integration vector DNA (e.g., does not grow on spectinomycin nor phleomycin plates). These colonies have been deleted for the targeted protease gene in the chromosomal DNA and no longer carry the pCRM-Ts Phleo plasmid.

This method was used to create the various BG6000 and BG6100 strains referred to below.

The strains bearing the protease gene deletions of interest are sequenced to verify excision of protease gene sequences. Following verification, the deletion mutants are then transformed with chromosomal DNA from B. subtilis strain EL534 to make the various NprE protease production strains. The strains are then transferred for production using a large-scale fermentor as described in Example 4. The protease composition of the broth is assessed by measuring protease activity and by SDS-PAGE analysis.

The strains of interest are denoted by the number of protease gene knock-outs. For example, the term 2-delete strain refers to B. subtilis bearing the deletion of the homologous aprE (serine protease/alkaline protease) and nprE (extracellular neutral metalloprotease) genes.

2-delete (aka SC6.1)
ΔaprE (serine alkaline protease)
ΔnprE (extracellular neutral metalloprotease)
   3-delete (aka BG6100)
ΔaprE (serine alkaline protease)
ΔnprE (extracellular neutral metalloprotease)
Δvpr (minor extracellular serine protease)
   4-delete (aka BG6101)
ΔaprE (serine alkaline protease)
ΔnprE (extracellular neutral metalloprotease)
Δepr (minor extracellular serine protease)
Δvpr (minor extracellular serine protease)
   5-delete (aka BG3934)
ΔaprE (serine alkaline protease)
ΔnprE (extracellular neutral metalloprotease)
Δepr (minor extracellular serine protease)
ΔispA (major intracellular serine protease)
Δbpr (serine protease)
   6-delete (aka BG6000)
ΔaprE (serine alkaline protease)
ΔnprE (extracellular neutral metalloprotease)
Δepr (minor extracellular serine protease)
ΔispA (major intracellular serine protease)
Δbpr (serine protease)
Δvpr (minor extracellular serine protease)
   8-delete (aka BG6003)
ΔaprE (serine alkaline protease)
ΔnprE (extracellular neutral metalloprotease)
Δepr (minor extracellular serine protease)
ΔispA (major intracellular serine protease)
Δbpr (serine protease)
Δvpr (minor extracellular serine protease)
ΔwprA (cell wall associated protease)
Δmpr-ybfJ (extracellular metalloprotease)

### Example 7

### NprE Protease Production in B. subtilis Host Strains Bearing Two Protease Gene Deletions

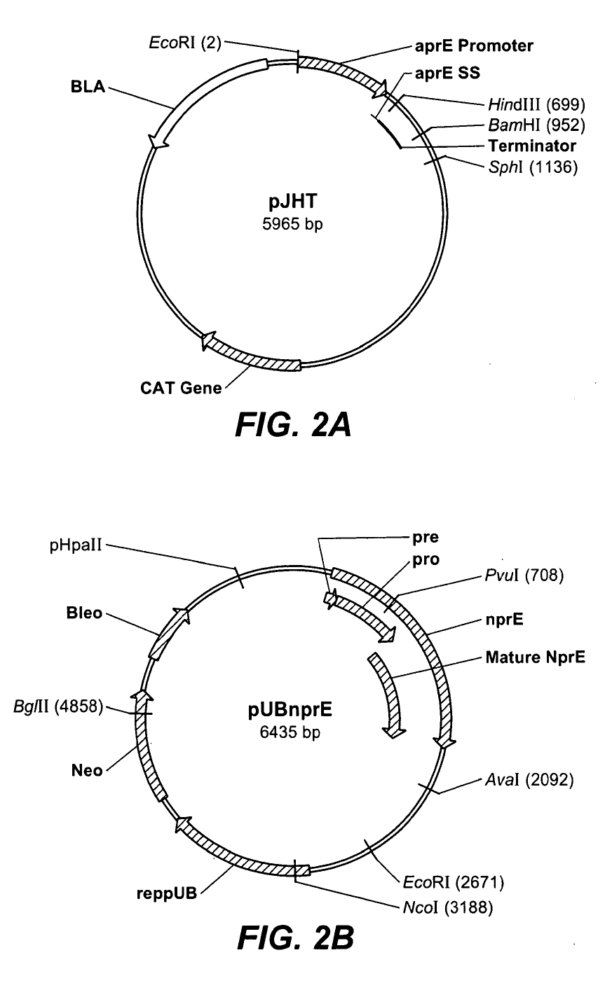

This example describes the production of NprE in a B. subtilis host strain engineered to lack two endogenous proteases (ΔaprE, ΔnprE), EL534 and EL535. Maps for plasmids pJHT and pUBnprE are provided in FIG. 2.

### Materials:

pJHT plasmad
pUB-nprE plasmad
Primers EL-755, EL-818, EL-819, and EL-820 (Operon Biotechnologies, Inc.)
SC6.1 Bacillus competent cells
KOD Hot Start DNA polymerase (Novagen)
QIAquick PCR purification kit (Qiagen)
pJM102 plasmid
TempliPhi Amplification Kit (GE Healthcare)
BG3594 competent cells (ΔaprE, ΔnprE, oppA, ΔspoIIE, degUHy32, ΔamyE::(xylR,pxylA-comK))

Primer Sequences:
(SEQ ID NO: 8)
EL-755:
CGTCTTCAAG AATTCCTCCA TTTTCTTCTGC
(SEQ ID NO: 9)
EL-818:
CAGACAATTT CTTACCTAAA CCCACTCTTT ACCCTCTCCT
TTTAAAAAAA TTCAG
(SEQ ID NO: 10)
EL-819:
CTGAATTTTT TTAAAAGGAG AGGGTAAAGA GTGGGTTTAG
GTAAGAAATT GTCTG
(SEQ ID NO: 11)
EL-820:
GCTTATGGAT CCGATCATGG TGAAGCCACT GTG

### Method:

To construct an integrating vector containing the aprE promoter (from Bacillus subtilis) with the nprE gene (from B. amyloliquefaciens) two separate PCR reactions were carried out.

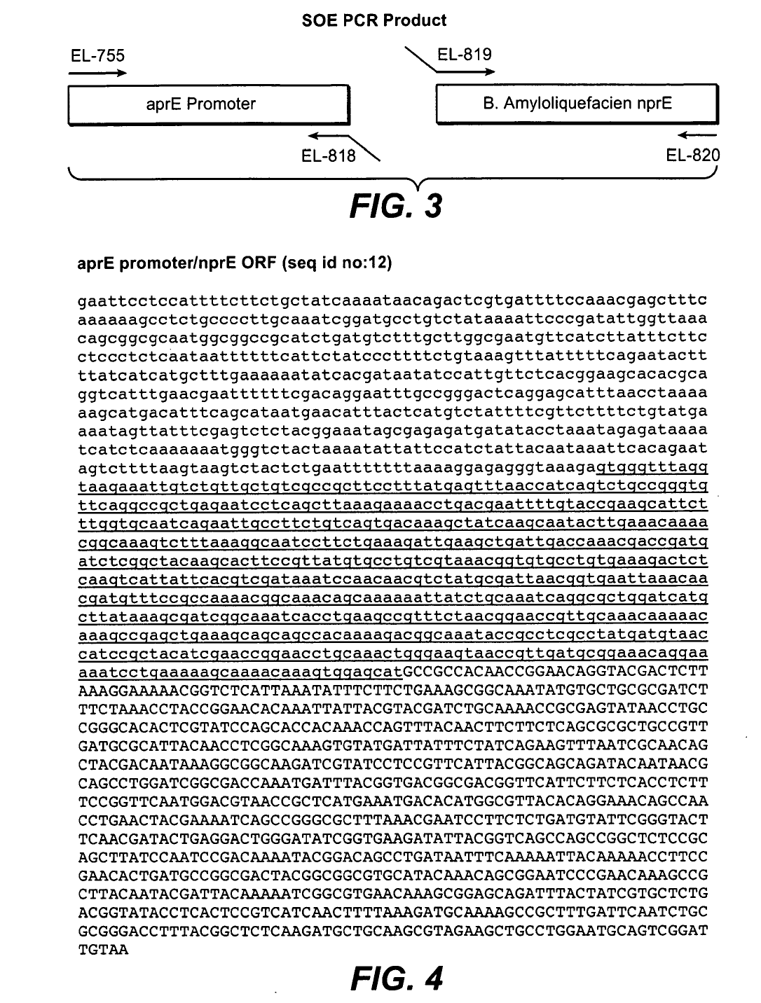

The first PCR reaction as illustrated in FIG. 3 involved amplifying the aprE promoter from plasmid, pJHT. In this reaction, the following reagents were combined: 1 μl pJHT plasmid (50 ng/μl), 1 μl Primer EL-755 (25 uM), 1 μl Primer EL-818 (25 μM), 10 μl 10× KOD buffer, 10 μl dNTP (2 mM), 4 μl MgSO4 (25 mM), 1 ul KOD Hot Start DNA polymerase, and 72 μl autoclaved Milli-Q water to provide a total reaction volume of 100 μl. The PCR cycles were: 95° C. for 2 minutes (1^{st} cycle only), followed by 28 cycles of 95° C. for 30 seconds, 54° C. for 30 seconds, and 72° C. for 16 seconds.

The second PCR reaction involved amplifying the nprE gene from plasmid pUB-nprE. In this reaction, the following reagents were combined: 1 μl pUB-nprE plasmid (50 ng/ul), 1 μl Primer EL-819 (25 uM), 1 μl Primer EL-820 (25 uM), 10 μl 10×KOD buffer, 10 μl dNTP (2 mM), 4 μl MgSO4 (25 mM), 1 μl KOD Hot Start DNA polymerase, and 72 μl autoclaved Milli-Q water to provide a total reaction volume of 100 ul.

The PCR cycles were: 95° C. for 2 minutes (1^{st} cycle only), followed by 28 cycles of 95° C. for 30 seconds, 56° C. for 30 seconds, and 72° C. for 40 seconds.

After amplifying each fragment, the PCR products were purified using the QIAquick PCR. The PCR Splice Overlap Extension (SOE) reaction was then used to join the two separate DNA fragments together. In the SOE reaction, the following reagents were combined: 1 μl aprE promoter DNA fragment, 1 μl B. amyloliquefacien nprE gene fragment, 1 μl Primer EL-755 (25 □M), 1 μl Primer EL-820 (25 μM), 10 μl 10×KOD buffer, 10 μl dNTP (2 mM), 4 μl MgSO4 (25 mM), 1 μl KOD Hot Start DNA polymerase, and 69 μl Milli-Q water (autoclaved) to provide a total reaction volume of 100 μl.

The PCR cycles were: 95° C. for 2 minutes (1^{st} cycle only), followed by 28 cycles of 95° C. for 30 seconds, 58° C. for 30 seconds, and 72° C. for 55 seconds.

The PCR fusion fragment of aprE promoter—B. amyloliquefaciens nprE gene was digested with EcoRI and BamHI restriction endonucleases. The pJM102 vector was digested with EcoRI and BamHI restriction endonucleases. The restriction endonuclease digested aprE promoter—B. amyloliquefaciens nprE fragment was then ligated with the restriction endonuclease digested pJM102 vector. TempliPhi rolling circle amplification was then used to generate large amounts of ligated DNA material for increased Bacillus transformation efficiency according to manufacturer's protocol. Specifically, 1 μl DNA ligation mixture, 5 μl TempliPhi Sample Buffer, 5 μl TempliPhi Reaction Buffer, and 0.20 μl TempliPhi Enzyme Mix in an 11 μl total reaction were incubated at 30° C. for 3 hours. The TempliPhi mixture was diluted by addition of 100 μl autoclaved Milli-Q water, and briefly vortexed.

Two μl of diluted TempliPhi material was transformed into BG3594-comK competent cells and plasmids that integrated into the aprE locus of the chromosome were selected by using LA+5 ppm chloramphenicol +1.6% skim milk plates. Transformants able to grow and produce a halo on the LA+5 ppm chloramphenicol +1.6% skim milk plates were considered to contain the integrated plasmid, resulting in the creation of strain EL534. Chromosomal DNA of strain EL534 was extracted and the integrated aprE promoter—B. amyloliquefaciens nprE gene fragment was then PCR amplified and sequenced to confirm its identity. The sequence of nucleic acid fragment including the aprE promoter fused to the nprE open reading frame is provided in FIG. 4 (SEQ ID NO:12). Afterwards, the strain was amplified using LA+25 ppm chloramphenicol +1.6% skim milk plates to obtain higher NprE protein expression levels, resulting in strain EL535.

### Example 8

### NprE Protease Production in B. subtilis Host Strains Bearing Three Protease Gene Deletions

This example describes the production of NprE in a B. subtilis host strain engineered to lack three endogenous proteases (ΔaprE, ΔnprE, Δvpr), EL549 and EL552.

### Materials:

Chromosomal DNA of EL534
BG6100 competent cells (ΔaprE, ΔnprE, Δvpr, oppA, ΔspoIIE, degUHy32,
ΔamyE::(xylR,pxylA-comK))

### Method:

One hundred 100 μl of BG6100 competent cells are transformed by adding 1 μl of EL534 chromosomal DNA (100 ng/μl). The transformed cells are then selected on LA+5 ppm chloramphenicol +1.6% skim milk plates. After picking a transformant that is capable of growing on 5 ppm chloramphenicol and producing a halo on skim milk plates, a 5 ml LB+5 ppm chloramphenicol tube is inoculated and grown overnight at 37° C. for chromosomal DNA extraction. Using the extracted chromosomal DNA, a second round of transformation into BG6100 competent cells is done to ensure the Bacillus chromosome contains all 3 protease deletions. Transformants able to grow and produce a halo on the LA+5 ppm chloramphenicol +1.6% skim milk plates are considered to contain the integrated plasmid, resulting in the creation of strain EL549. Afterwards, the strain is amplified using LA+25 ppm chloramphenicol +1.6% skim milk plates to obtain higher heterologous NprE protein expression levels, resulting in strain EL552.

### Example 9

### NprE Protease Production in B. subtilis Host Strains Bearing Four Protease Gene Deletions

This example describes the production of NprE in a B. subtilis host strain engineered to lack four endogenous proteases (ΔaprE, ΔnprE, Δepr, Δvpr), EL550 and EL553.

### Materials:

Chromosomal DNA of EL534
BG6101 competent cells (ΔaprE, ΔnprE, Δepr, Δvpr, oppA, ΔspoIIE, degUHy32, ΔamyE::(xylR,pxylA-comK))

### Method:

One hundred μl of BG6101 competent cells are transformed by adding 1 μl of EL534 chromosomal DNA (100 ng/μl). The transformed cells are then selected on LA+5 ppm chloramphenicol +1.6% skim milk plates. After picking a transformant that is capable of growing on 5 ppm chloramphenicol and producing a halo on skim milk plates, 5 ml LB+5 ppm chloramphenicol tubes are inoculated and grown overnight at 37° C. for chromosomal DNA extraction. Using the extracted chromosomal DNA, a second round of transformation into BG6101 competent cells is done to ensure the chromosome contains all 4 protease deletions. Transformants able to grow and produce a halo on the LA+5 ppm chloramphenicol +1.6% skim milk plates are considered to contain the integrated plasmid, resulting in the creation of strain EL550. Afterwards, the strain is amplified using LA+25 ppm chloramphenicol +1.6% skim milk plates to obtain higher heterologous NprE protein expression levels, resulting in strain EL553.

### Example 10

### NprE Protease Production in B. subtilis Host Strains Bearing Five Protease Gene Deletions

This example describes the production of NprE in a B. subtilis host strain engineered to lack five endogenous proteases (ΔaprE, ΔnprE, Δepr, ΔispA, Δbpf), EL543 and EL546.

### Materials:

Chromosomal DNA of EL534
BG3934 competent cells (ΔaprE, ΔnprE, Δepr, ΔispA, Δbpf, oppA, ΔspoIIE, degUHy32, ΔamyE::(xylR,pxylA-comK))

### Method:

One hundred μl of BG3934-comK competent cells were transformed by addition of 1 μl of EL534 chromosomal DNA (100 ng/μl). The transformed cells were then selected on LA+5 ppm chloramphenicol +1.6% skim milk plates. After picking a transformant capable of growing on 5 ppm chloramphenicol and producing a halo on skim milk plates, 5 ml LB+5 ppm chloramphenicol tubes were inoculated and grown overnight at 37° C. for chromosomal DNA extraction. Using the extracted chromosomal DNA, a second round of transformation into BG3934 competent cells is done to ensure the chromosome contains all five protease deletions. Transformants able to grow and produce a halo on the LA+5 ppm chloramphenicol +1.6% skim milk plates are considered to contain the integrated plasmid, resulting in the creation of strain EL543. Afterwards, the strain was amplified using LA+25 ppm chloramphenicol +1.6% skim milk plates to obtain higher heterologous NprE protein expression levels, resulting in strain EL546.

### Example 11

### NprE Protease Production in B. subtilis Host Strains Bearing Six Protease Gene Deletions

This example describes the production of NprE in a B. subtilis host strain engineered to lack six endogenous proteases (ΔaprE, ΔnprE, Δepr, ΔispA, Δbpf, Δvpr), EL544 and EL547.

### Materials:

Chromosomal DNA of EL534
BG6000 competent cells (ΔaprE, ΔnprE, Δepr, ΔispA, Δbpf, Δvpr, oppA, ΔspoIIE, degUHy32, ΔamyE::(xylR,pxylA-comK))

### Method:

One hundred μl of BG6000 competent cells were transformed by addition of 10 of EL534 chromosomal DNA (100 ng/μl). The transformed cells were then selected on LA+5 ppm chloramphenicol +1.6% skim milk plates. After picking a transformant capable of growing on 5 ppm chloramphenicol and producing a halo on skim milk plates, a 5 ml LB+5 ppm chloramphenicol tube is inoculated and grown overnight at 37° C. for chromosomal DNA extraction. Using the extracted chromosomal DNA, a second round of transformation is done into BG6000 competent cells to ensure the chromosome contains all six protease deletions. Transformants able to grow and produce a halo on the LA+5 ppm chloramphenicol +1.6% skim milk plates are considered to contain the integrated plasmid, resulting in the creation of strain EL544. Afterwards, the strain was amplified using LA+25 ppm chloramphenicol +1.6% skim milk plates to obtain higher heterologous NprE protein expression levels, resulting in strain EL547.

### Example 12

### NprE Protease Production in B. subtilis Host Strains Bearing Eight Protease Gene Deletions

This example describes the production of NprE in a B. subtilis host strain engineered to lack eight endogenous proteases (ΔaprE, ΔnprE, Δepr, ΔispA, Δbpf, Δvpr, ΔwprA, Δmpr-ybfJ), EL545 and EL548.

### Materials:

Chromosomal DNA of EL534
BG6003 competent cells (ΔaprE, ΔnprE, Δepr, ΔispA, Δbpf, Δvpr, ΔwprA, Δmpr-ybfJ, oppA, ΔspoIIE, degUHy32, ΔamyE::(xylR,pxylA-comK))

### Method:

One hundred μl of BG6003 competent cells were transformed by addition of 10 of EL534 chromosomal DNA (100 ng/μl). The transformed cells were then selected on LA+5 ppm chloramphenicol +1.6% skim milk plates. After picking a transformant capable of growing on 5 ppm chloramphenicol and producing a halo on skim milk plates, a 5 ml LB+5 ppm chloramphenicol tube was inoculated and grown overnight at 37° C. for chromosomal DNA extraction. Using the extracted chromosomal DNA, a second round of transformation is done into BG6003 competent cells to ensure the chromosome contains all eight protease deletions. Transformants able to grow and produce a halo on the LA+5 ppm chloramphenicol +1.6% skim milk plates are considered to contain the integrated plasmid, resulting in the creation of strain EL545. Afterwards, the strain was amplified using LA+25 ppm chloramphenicol +1.6% skim milk plates to obtain higher heterologous NprE protein expression levels, resulting in strain EL548.

### Example 13

### Shake Flask Evaluations

This example describes the method used to evaluate expression of heterologous NprE in various B. subtilis host strains.

### Materials:

Bacillus strains EL535, EL546, EL547, EL552, EL553 and EL548
LA+25 ppm chloramphenicol +1.6% skim milk plates
LB+25 ppm chloramphenicol
Shake flask evaluation media +25 ppm chloramphenicol
1N Hydrochloric acid
NuPage 10% Bis-Tris Gel (Invitrogen)
Novex 2× Tris-Glycine SDS Sample Buffer (Invitrogen)
NuPage 20×MES SDS Running Buffer (Invitrogen)
Novex XCell SureLock Mini-Cell (Invitrogen)
SimplyBlue SafeStain (Invitrogen)
   Shake Flask Media—Part 1 (using 1 liter bottle)
0.03 g MgSO₄ (anhydrous)
0.22 g K₂HPO₄
21.32 g Na₂HPO₄β*7H2O
6.1 g NaH₂PO₄*H20
3.6 g Urea
Bring up volume up with Milli-Q water to 500 ml
   Shake Flask Media—Part 2 (using 1 liter bottle)
7 g Cargill soymeal
Bring up volume up with Milli-Q water to 400 ml
   Shake Flask Media—Stock solution
350 g Maltrin M150
210 g Glucose
Bring volume up with Milli-Q water to 1 liter
   Allow to cool, combine Part 1 with Part 2, and add 100 ml Stock solution.

### Method:

Glycerol stocks of various B. subtilis strains expressing NprE were streaked onto LA+25 ppm chloramphenicol +1.6% skim milk plates. Plates were incubated overnight at 37° C. The next day, 2 ml LB+25 ppm chloramphenicol tubes were inoculated with a single colony of each strain to be analyzed. Cultures were grown at 37° C. with 225 rpm shaking for 6 hours. A 1:1000 dilution was made of the pre-culture into 25 ml of shake flask evaluation media +25 ppm chloramphenicol. Cultures were grown at 37° C. with 225 rpm for 40 hours. After 40 hours, cells were harvested by centrifugation. The cell supernatants were transferred to separate containers.

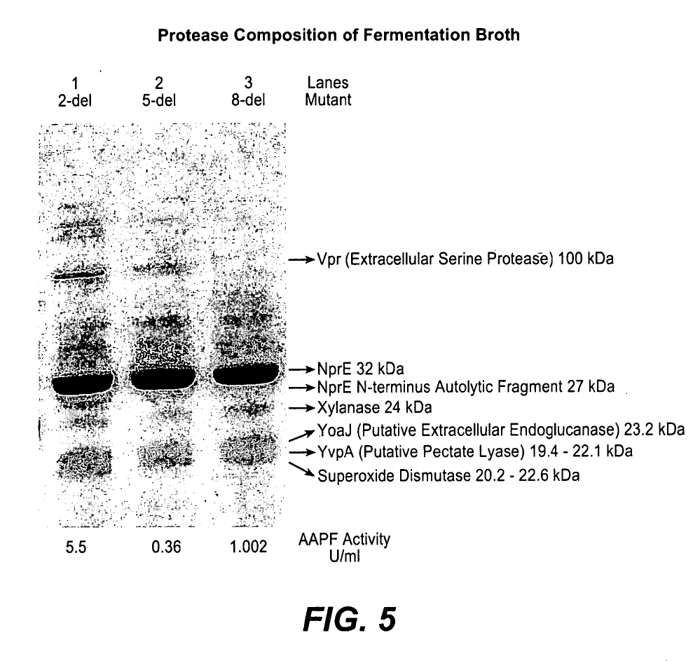

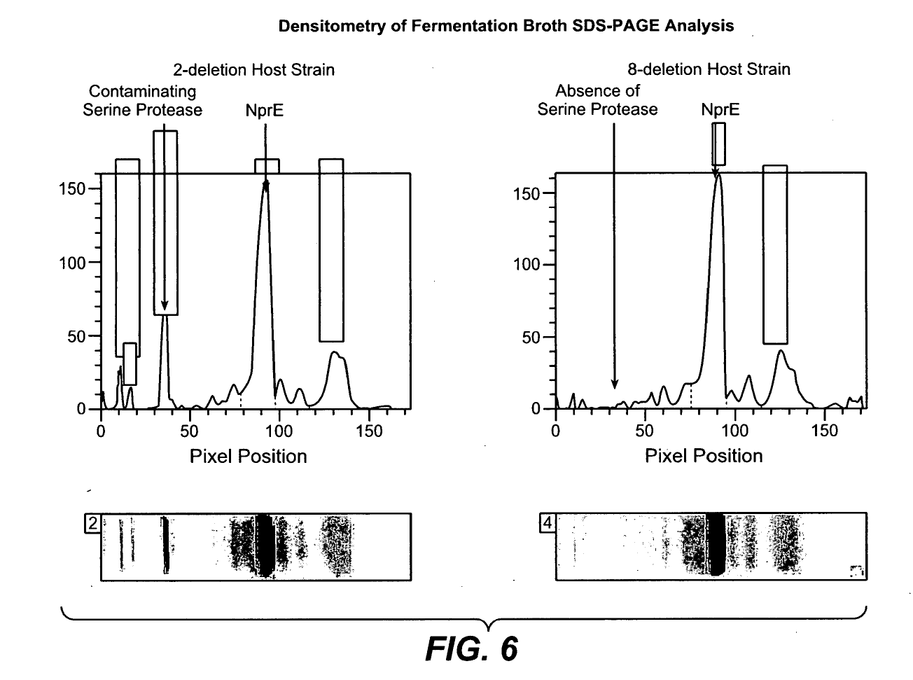

To prepare cell supernatant samples for SDS protein gel analysis, 100 μl cell supernatant was mixed with 25 μl ice, cold 1N hydrochloric acid, followed by incubation on ice for 10 minutes. Then 62.5 μl Novex 2× Tris-Glycine SDS Sample Buffer was added, followed by incubation at room temperature for 10 minutes. A NuPage 10% Bis-Tris Gel was set up in a Novex Xcell SureLock Mini-Cell using NuPage MES SDS Running Buffer (according to manufacturer's protocol). Cell supernatant samples were loaded into wells of the NuPage 10% Bis-Tris Gel, and run according to the manufacturer's protocol. The SDS protein gel was rinsed with water and stained with SimplyBlue SafeStain (according to manufacturer's protocol). As shown in FIG. 5 and FIG. 6, the contaminating Vpr protease was present in the supernatants of the 2-delete strain, but not in supernatants of the 8-delete strain.

In addition, assessment of serine protease activity against AAPF using the methods of Example 1, indicated that serine protease activity decreased with increasing enzyme deletions (Table 13-1).

TABLE 13-1
Serine Protease Activity

| Host Strain | AAPF Activity | Lane* |
|---|---|---|
| 2-delete | 5.5 U/ml | 1 |
| 5-delete | 0.36 U/ml | 2 |
| 8-delete | 0.002 U/ml | 3 |

*Lane numbers refer to the protein gels shown in FIG. 5.

In short, deletion of genes encoding serine proteases resulted in the production of a neutral metalloprotease production strain having a serine protease to metalloprotease ratio of less than 1%.

All patents and publications mentioned in the specification are indicative of the levels of those skilled in the art to which the invention pertains. All patents and publications are herein incorporated by reference to the same extent as if each individual publication was specifically and individually indicated to be incorporated by reference. However, the citation of any publication is not to be construed as an admission that it is prior art with respect to the present invention.

Having described exemplary embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the concepts herein disclosed may be resorted to by those skilled in the art, and that such modifications and variations are considered to be within the scope of this invention as defined by the appended claims

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

We claim:

1. A method for producing a neutral metalloprotease comprising:
i) providing a Bacillus host cell lacking an endogenous serine alkaline protease enzyme (AprE), an endogenous extracellular neutral metalloprotease enzyme (NprE), and an endogenous minor extracellular serine protease enzyme (Vpr);
ii) transforming said Bacillus host cell with a nucleic acid encoding a heterologous NprE enzyme in operable combination with a promoter to provide a transformed host cell; and
iii) cultivating said transformed host cell under conditions suitable for the production of said heterologous NprE enzyme, such that said heterologous NprE enzyme is produced.

2. The method of claim 1, further comprising recovering said produced heterologous NprE enzyme.

3. The method of claim 1, wherein said Bacillus host cell is B. subtilis.

4. The method of claim 1, wherein said Bacillus host cell further lacks an endogenous minor extracellular serine protease enzyme (Epr).

5. The method of claim 1, wherein said Bacillus host cell further lacks an endogenous major intracellular serine protease enzyme (IspA), and/or an endogenous bacillopeptidase F enzyme (Bpr).

6. The method of claim 1, wherein said Bacillus host cell lacks an endogenous cell wall associated protease enzyme (WprA), and/or an endogenous extracellular metalloprotease enzyme (Mpr).

7. The method of claim 1, wherein said heterologous NprE enzyme is a Bacillus amyloliquefaciens NprE enzyme or a variant thereof.

8. The method of claim 7, wherein said Bacillus amyloliquefaciens NprE variant has an amino acid sequence comprising a substitution in at least one position selected from positions equivalent to positions 1, 3, 4, 5, 6, 11, 12, 13, 14, 16, 21, 23, 24, 25, 31, 32, 33, 35, 36, 38, 44, 45, 46, 47, 48, 49, 5, 51, 54, 55, 58, 59, 6, 61, 62, 63, 65, 66, 69, 7, 76, 85, 86, 87, 88, 9, 91, 92, 96, 97, 98, 99, 1, 12, 19, 11, 111, 112, 113, 115, 117, 119, 127, 128, 129, 13, 132, 135, 136, 137, 138, 139, 14, 146, 148, 151, 152, 153, 154, 155, 157, 158, 159, 161, 162, 169, 173, 178, 179, 18, 181, 183, 184, 186, 19, 191, 192, 196, 198, 199, 2, 22, 23, 24, 25, 21, 211, 214, 215, 216, 217, 218, 219, 22, 221, 222, 223, 224, 228, 229, 237, 239, 24, 243, 244, 245, 248, 252, 253, 26, 261, 263, 264, 265, 267, 269, 27, 273, 277, 28, 282, 283, 284, 285, 286, 288, 289, 29, 292, 293, 296, 297, and 299 of the amino acid sequence set forth in SEQ ID NO:3.

9. The method of claim 8, wherein said Bacillus amyloliquefaciens NprE variant has an amino acid sequence comprising at least one substitution selected from T4C, T4E, T4H, T4I, T4K, T4L, T4M, T4N, T4P, T4R, T4S, T4V, T4W, T4Y, G12D, G12E, G121, G12K, G12L, G12M, G12Q, G12R, G12T, G12V, G12W, K13A, K13C, K13D, K13E, K13F, K13G, K13H, K13I, K13L, K13M, K13N, K13Q, K13S, K13T, K13V, K13Y, T14F, T14G, T14H, T14I, T14K, T14L, T14M, T14P, T14Q, T14R, T14S, T14V, T14W, T14Y, S23A, S23D, S23F, S23G, S23I, S23K, S23L, S23M, S23N, S23P, S23Q, S23R, S23S, S23T, S23V, S23W, S23Y, G24A, G24D, G24F, G24G, G24H, G24I, G24K, G24L, G24M, G24N, G24P, G24R, G24S, G24T, G24V, G24W, G24Y, K33H, Q45C, Q45D, Q45E, Q45F, Q45H, Q45I, Q45K, Q45L, Q45M, Q45N, Q45P, Q45R, Q45T, Q45W, N46A, N46C, N46E, N46F, N46G, N46H, N46I, N46K, N46L, N46M, N46P, N46Q, N46R, N46S, N46T, N46V, N46W, N46Y, R47E, R47K, R47L, R47M, R47Q, R47S, R47T, Y49A, Y49C, Y49D, Y49E, Y49F, Y49H, Y49I, Y49K, Y49L, Y49N, Y49R, Y49S, Y49T, Y49V, Y49W, N50D, N50F, N50G, N50H, N50I, N50K, N50L, N50M, N50P, N50Q, N50R, N50W, N50Y, T54C, T54D, T54E, T54F, T54G, T54H, T54I T54K, T54L, T54M, T54N, T54P, T54Q, T54R, T54S, T54V, T54W, T54Y, S58D, S58H, S58I, S58L, S58N, S58P, S58Q, T59A, T59C, T59E, T59G, T59H, T59I, T59K, T59L T59M, T59N, T59P, T59Q, T59R, T59S, T59V, T59W, T60D, T60F, T60I, T60K, T60L, T60N, T60Q, T60R, T60V, T60W, T60Y, T65C, T65E, T65F, T65H, T65I, T65K, T65L, T65M, T65P, T65Q, T65R, T65V, T65Y, S66C, S66D, S66E, S66F, S66H, S66I, S66K, S66L, S66N, S66P, S66Q, S66R, S66T, S66V, S66W, S66Y, Q87A, Q87D, Q87E, Q87H, Q87I, Q87K, Q87L, Q87M, Q87N, Q87R, Q87S, Q87T, Q87V, Q87W, N90C, N90D, N90E, N90F, N90G, N90H, N90K, N90L, N90R, N90T, N96G, N96H, N96K, N96R, K97H, K97Q, K97W, K100A, K100D, K100E, K100F, K100H, K100N, K100P, K100Q, K100R, K100S, K100V, K100Y, R110A, R110C, R110E, R110H, R110K, R110L, R110M, R110N, R110Q, R110S, R110Y, D119E, D119H, D1191, D119L, D119Q, D119R, D119S, D119T, D119V, D119W, G128C, G128F, G128H, G128K, G128L, G128M, G128N, G128Q, G128R, G128W, G128Y, S129A, S129C, S129D, S129F, S129G, S129H, S129I, S129K, S129L, S129M, S129Q, S129R, S129T, S129V, S129W, S129Y, F130I, F130K, F130L, F130M, F130Q, F130R, F130T, F130V, F130Y, S135P, G136I, G136L, G136P, G136V, G136W, G136Y, S137A, M138I, M138K, M138L, M138Q, M138V, D139A, D139C, D139E, D139G, D139H, D1391, D139K, D139L, D139M, D139P, D139R, D139S, D139V, D139W, D139Y, V140C, Q151I, E152A, E152C, E152D, E152F, E152G, E152H, E152L, E152M, E152N, E152R, E152S, E152W, N155D, N155K, N155Q, N155R, D178A, D178C, D178G, D178H, D178K, D178L, D178M, D178N, D178P, D178Q, D178R, D178S, D178T, D178V, D178W, D178Y, T179A, T179F, T179H, T179I, T179K, T179L, T179M, T179N, T179P, T179Q, T179R, T179S, T179V, T179W, T179Y, E186A, E186C, E186D, E186G, E186H, E186K, E186L, E186M, E186N, E186P, E186Q, E186R, E186S, E186T, E186V, E186W, E186Y, V190H, V190I, V190K, V190L, V190Q, V190R, S191F, S191G, S191H, S1911, S191K, S191L, S191N, S191Q, S191R, S191W, L198M, L198V, S199C, S199D, S199E, S199F, S199I, S199K, S199L, S199N, S199Q, S199R, S199V, Y204H, Y204T, G205F, G205H, G205L, G205M, G205N, G205R, G205S, G205Y, K211A, K211C, K211D, K211G, K211M, K211N, K211Q, K211R, K211S, K211T, K211V, K214A, K214C, K214E, K214I, K214L, K214M, K214N, K214Q, K214R, K214S, K214V, L216A, L216C, L216F, L216H, L216Q, L216R, L216S, L216Y, N218K, N218P, T219D, D220A, D220E, D220H, D220K, D220N, D220P, A221D, A221E, A221F, A221I, A221K, A221L, A221M, A221N, A221S, A221V, A221Y, G222C, G222H, G222N, G222R, Y224F, Y224H, Y224N, Y224R, T243C, T243G, T243H, T243I, T243K, T243L, T243Q, T243R, T243W, T243Y, K244A, K244C, K244D, K244E, K244F, K244G, K244L, K244M, K244N, K244Q, K244S, K244T, K244V, K244W, K244Y, V260A, V260D, V260E, V260G, V260H, V260I, V260K, V260L, V260M, V260P, V260Q, V260R V260S, V260T, V260W, V260Y, Y261C, Y261F, Y261I, Y261L, T263E, T263F, T263H, T263I, T263L, T263M, T263Q, T263V, T263W, T263Y, S265A, S265C, S265D, S265E, S265K, S265N, S265P, S265Q, S265R, S265T, S265V, S265W, K269E, K269F, K269G, K269H, K269I, K269L, K269M, K269N, K269P, K269Q, K269S, K269T, K269V, K269W, K269Y, A273C, A273D, A273H, A273I, A273K, A273L, A273N, A273Q, A273R, A273Y, R280A, R280C, R280D, R280E, R280F, R280G, R280H, R280K, R280L, R280M, R280S, R280T, R280V, R280W, R280Y, L282F, L282G, L282H, L282I, L282K, L282M, L282N, L282Q, L282R, L282V, L282Y, S285A, S285C, S285D, S285E, S285K, S285P, S285Q, S285R, S285W, Q286A, Q286D, Q286E, Q286K, Q286P, Q286R, A289C, A289D, A289E, A289K, A289L, A289R, A293C, A293R, N296C, N296D, N296E, N296K, N296R, N296V, A297C, A297K, A297N, A297Q, A297R, and G299N, of the amino acid sequence set forth in SEQ ID NO:3.

10. The method of claim 8, wherein said Bacillus amyloliquefaciens NprE variant has an amino acid sequence comprising multiple substitutions selected from S129I/F130L/D220P, M138L/V190I/D220P, and S120I/F130L/M138L/V190I/D220P.

11. The method of claim 1, wherein said neutral metalloprotease has at least about 45% amino acid identity with the neutral metalloprotease comprising the amino acid sequence set forth as SEQ ID NO:3.

12. The heterologous NprE enzyme produced by the method set forth in claim 1.

13. An isolated NprE enzyme having at least about 45% amino acid identity with a neutral metalloprotease comprising the amino acid sequence set forth in SEQ ID NO:3, or variants thereof.

14. A composition comprising the NprE enzyme or variant thereof set forth in claim 13, wherein said composition is essentially devoid of Bacillus serine protease enzyme (AprE) contamination.

15. The composition of claim 14, wherein said AprE contamination comprises less than about 1% by weight as compared to the NprE or variant thereof of said composition.

16. The composition of claim 15, wherein said AprE contamination comprises less than about 0.50 U/ml serine protease activity.

17. The composition of claim 16, wherein said AprE contamination comprises less than about 0.05 U/ml serine protease activity.

18. The composition of claim 17, wherein said AprE contamination comprises less than about 0.005 U/ml serine protease activity.

19. The composition of claim 14, wherein said composition is a cleaning composition.

20. The composition of claim 19, wherein said cleaning composition is a detergent.

21. The composition of claim 14, further comprising at least one additional enzyme or enzyme derivative selected from the group of amylases, lipases, mannanases, pectinases, cutinases, oxidoreductases, hemicellulases, and cellulases.

22. The composition of claim 14, wherein said composition comprises at least about 0.0001 weight percent of said NprE enzyme or variant thereof.

23. The composition of claim 22, wherein said composition comprises from about 0.001 to about 0.5 weight percent of said NprE enzyme or variant thereof.

24. The composition of claim 14, further comprising at least one adjunct ingredient.

25. The composition of claim 14, further comprising a sufficient amount of a pH modifier to provide the composition with a neat pH of from about 3 to about 5, the composition being essentially free of materials that hydrolyze at a pH of from about pH 3 to about pH 5.

26. The composition of claim 25, wherein said materials that hydrolyze at a pH of from about pH 3 to about pH 5 comprise at least one surfactant.

27. The composition of claim 26, wherein said surfactant is a sodium alkyl sulfate surfactant comprising an ethylene oxide moiety.

28. The composition of claim 14, wherein said composition is a liquid.

29. A method for cleaning, comprising contacting a surface and/or an article comprising a fabric with the cleaning composition of claim 19.

30. The method of claim 29, further comprising the step of rinsing the surface and/or material after contacting said surface or material with said cleaning composition.

31. The composition of claim 14, wherein said composition is an animal feed.

32. The composition of claim 14, wherein said composition is a textile and/or a leather processing composition.

33. A Bacillus host cell lacking an endogenous serine alkaline protease enzyme (AprE), an endogenous extracellular neutral metalloprotease enzyme (NprE), and an endogenous minor extracellular serine protease enzyme (Vpr), wherein said host cell is transformed with a nucleic acid encoding a heterologous NprE enzyme in operable combination with a promoter.

34. The Bacillus host cell of claim 33, wherein said Bacillus host cell is B. subtilis.

35. The Bacillus host cell of claim 33, further lacking an endogenous minor extracellular serine protease enzyme (Epr).

36. The Bacillus host cell of claim 35, further lacking an endogenous major intracellular serine protease enzyme (IspA), and/or an endogenous bacillopeptidase F enzyme (Bpr).

37. The Bacillus host cell of claim 36, wherein said Bacillus host cell is B. subtilis.

38. The Bacillus host cell of claim 36, further lacking an endogenous cell wall associated protease enzyme (WprA), and/or an endogenous extracellular metalloprotease enzyme (Mpr).

39. The Bacillus host cell of claim 38, wherein said Bacillus host cell is B. subtilis.

40. The Bacillus host cell of claim 33, wherein said heterologous NprE enzyme is a Bacillus amyloliquefaciens NprE enzyme or a variant thereof.

41. An isolated Bacillus host cell lacking an endogenous serine alkaline protease enzyme (AprE), an endogenous extracellular neutral metalloprotease enzyme (NprE), an endogenous minor extracellular serine protease enzyme (Vpr), an endogenous minor extracellular serine protease enzyme (Epr), an endogenous major intracellular serine protease enzyme (IspA), and an endogenous bacillopeptidase F enzyme (Bpr).

42. The Bacillus host cell of claim 41, wherein said host cell is B. subtilis.

43. An isolated Bacillus host cell lacking an endogenous serine alkaline protease enzyme (AprE), an endogenous extracellular neutral metalloprotease enzyme (NprE), an endogenous minor extracellular serine protease enzyme (Vpr), an endogenous minor extracellular serine protease enzyme (Epr), an endogenous major intracellular serine protease enzyme (IspA), an endogenous bacillopeptidase F enzyme (Bpr), an endogenous cell wall associated protease enzyme (WprA), and an endogenous extracellular metalloprotease enzyme (Mpr).

54. The Bacillus host cell of claim 43, wherein said host cell is B. subtilis.
